(19) United States
(12) Patent Application Publication
(10) Pub. No.: US 2006/0084064 A1
(43) Pub. Date: Apr. 20, 2006

(54) Endocan compositions and methods for the treatment of neoplasms

(76) Inventors: William C Aird, Brookline, MA (US); Xianjin Yi, Quincy, MA (US)

Correspondence Address: CLARK & ELBING LLP, 101 FEDERAL STREET, BOSTON, MA, 02110, US

(21) Appl. No.: 10/542,717

(22) PCT Filed: Jan. 22, 2004
(86) PCT No.: PCT/US04/01668

§ 371 Date: Dec. 1, 2005

Related U.S. Application Data
(60) Provisional application No. 60/441,872, filed on Jan. 22, 2003.

(51) Int. Cl. C12Q 1/68; G01N 33/574

(52) U.S. Cl. 435/6; 435/7.23

(57) ABSTRACT

The present invention features improved diagnostic and therapeutic methods for neoplasia treatment relating to endocan nucleic acid molecules and polypeptides. In addition, the invention features methods for monitoring the responsive of a tumor in a patient to therapy; methods for suppressing the growth of a neoplasm by inhibiting endocan expression or biological activity; methods for generating a promoter that directs expression of a heterologous nucleic acid sequence specifically to the endothelium of tumor blood vessels; and methods for using such promoters to deliver peptides to a tumor in a patient.

### BACKGROUND OF THE INVENTION

The present invention features improved diagnostic and therapeutic methods for neoplasia treatment relating to endocan nucleic acid molecules and polypeptides.

One of the major strategies in combating neoplasic growth has been to inhibit the formation of the network of blood vessels around a neoplasm. The vessels provide the neoplasm with essential nutrients and remove waste, and are, therefore, essential for neoplasm viability. Angiogenesis, which is the formation of new vessels from pre-existing vessels, plays a physiological role in development, the female reproductive cycle, and wound healing, and is a critical determinant of cancer progression and metastasis. To gain a molecular understanding of neoplasm angiogenesis, researchers have used a SAGE approach to identify novel tumor endothelial markers (TEMs) that are expressed in tumor endothelial cells, in the corpus luteum, and during the wound healing process. However, subsequent studies revealed the presence of TEMs in occasional endothelial cells of normal adult tissues and at high levels in the endothelium of embryonic tissues, suggesting that the previously identified TEMs are markers for neoangiogenesis and not limited to tumor angiogenesis.

There exists a need for markers that allow the physician or other practitioner to readily determine the responsiveness of a neoplasm in a patient to therapy.

### SUMMARY OF THE INVENTION

In general, the present invention features improved diagnostic and therapeutic methods for neoplasia treatment that utilize endocan nucleic acid molecules and polypeptides and related molecules.

We have discovered that endocan gene expression is a novel marker for tumor endothelium. Endocan (also known as “endothelial cell-specific molecule-1” or “ESM-1”) is a soluble dermatan sulfate proteoglycan (DSPG) that is secreted by cultured endothelial cells. Circulating levels of endocan were reported to be markedly elevated in patients with severe sepsis. Functional assays have revealed that endocan regulates LFA-1-intercellular adhesion molecule-1 interactions and may be involved in the regulation of leukocyte trafficking. Moreover, endocan has been shown to regulate the mitogenic activity of hepatocyte growth factor/scatter factor (HGF/SF).

The limited in vivo distribution of the endocan mRNA and protein indicates that monitoring endocan expression and the levels of endocan biological activity may be used as valuable marker for the level of tumor vasculature. Decreases in level of vasculature, in turn, provide an indication of the responsiveness of a tumor to therapy. Indicators of endocan gene expression further provide a valuable probe for identifying the mechanisms of tumor endothelial cell-specific gene regulation. Moreover, our finding that endocan is modulated by growth factor activity indicates that endocan is involved in autocrine and/or paracrine control of cell proliferation. We show, in mice, that the endocan gene is expressed predominantly within the endothelial lining of tumor microvessels. In addition, our data support a role for vascular endothelial growth factor (VEGF) in inducing endocan expression in tumor endothelium and a role for epidermal growth factor (EGF) in down-regulating endocan levels in normal tissues. Finally, endothelial-derived endocan plays a functional role in mediating tumor growth, and thus may serve as a therapeutic target in treating tumors, as well as a marker for monitoring the responsiveness of a tumor to therapy.

While there has been a major thrust in recent years to identify novel anti-angiogenic molecules that will inhibit blood vessel formation and thereby starve tumors of oxygen and essential nutrients, relatively little attention has been paid to understanding the molecular basis of tumor endothelial cell-specific phenotypes. Endothelial cells display marked heterogeneity under normal and pathophysiological conditions. Tumor endothelium differs from other types of endothelium at the level of structure, function, gene expression, and promoter activity. In theory, such properties may be exploited to deliver biologically active molecules to tumors.

Below we provide results demonstrating tumor endothelial cell-specific difference in function among promoters (e.g., Flt, Tie-2, vWF) that drive robust expression in endothelial cells. These studies dissect different endothelial-specific promoters and provide a better understanding of transcriptional regulation of gene expression in tumor endothelium. Our results allow the generation of non-naturally occurring promoters that direct high-level expression specifically to the endothelium of tumor blood vessels. Such non-naturally occurring promoters may be used, among other things, to deliver therapeutic products to the tumor.

In one aspect, the invention generally features a method of diagnosing a patient as having a neoplasia, the method involves detecting an endocan nucleic acid molecule or polypeptide in a patient sample, where detection of an endocan nucleic acid or polypeptide indicates that the patient has a neoplasia. In one embodiment, the method further involves detecting an increase in the level of expression of an endocan polypeptide in a patient sample relative to the level of endocan polypeptide present in a corresponding control sample from a normal individual. In another embodiment, the level of expression is determined in an immunological assay (e.g., by ELISA). In one preferred embodiment, the patient is asymptomatic.

In another aspect, the invention features a method of assessing the responsiveness of a neoplasm to a treatment regimen, the method involves determining the level of an endocan nucleic acid or polypeptide in a patient sample relative to the level in a reference sample, where an alteration in the nucleic acid or polypeptide level in the patient sample indicates the responsiveness of the neoplasm to a treatment regimen. In one embodiment, reference sample is derived from a healthy individual. In a preferred embodiment, the patient is being treated for a neoplasm. In another preferred embodiment, the reference sample is obtained from the patient prior to or during the course of the treatment regimen. In another embodiment, the alteration is an increase, and the increase indicates a decreased responsiveness of the neoplasm to a treatment regimen. In another embodiment, the alteration is a decrease, and the decrease indicates an increased responsiveness of the neoplasm to a treatment regimen.

In another aspect, the invention features a method of determining the prognosis of a patient having a neoplasm, the method involves detecting an alteration in the level of an endocan nucleic acid molecule or polypeptide in a patient sample relative to the level in a reference sample, wherein an alteration indicates the prognosis of the patient. In one embodiment, the reference sample is obtained from the patient prior to or during the course of the treatment regimen. In another embodiment, the alteration is an increase, and the increase indicates a poor prognosis. In yet another embodiment, alteration is a decrease, and the decrease indicates a good prognosis. In yet another embodiment, the level of expression is determined in an immunological or enzymatic assay.

In another aspect, the invention features a diagnostic kit for the identification of a neoplasm in a patient containing an endocan nucleic acid or amino acid sequence, or a fragment thereof.

In another aspect, the invention features a diagnostic kit for the identification of a neoplasm in a patient containing an anti-endocan antibody.

In yet another aspect, the invention features a method of treating or preventing a neoplasm, the method involves administering to a patient in need of such treatment an effective amount of a pharmaceutical composition containing an inhibitory endocan nucleic acid molecule or fragments thereof. In one embodiment, where the nucleic acid molecule is an antisense nucleic acid molecule that decreases the expression of an endocan nucleic acid molecule or polypeptide in a cell. In preferred embodiments, the inhibitory endocan nucleic acid molecule is a double stranded nucleic acid molecule, siRNA, or shRNA that decreases the expression of an endocan nucleic acid molecule or polypeptide.

In another aspect, the invention features a method of treating or preventing a neoplasm, the method involves administering to a patient in need of such treatment an effective amount of a pharmaceutical composition containing an inhibitor of endocan biological activity. In one embodiment, the inhibitor is an antibody or an antigen-binding fragment thereof that specifically binds endocan. In another embodiment, the antibody is a monoclonal antibody. In another embodiment, antibody or antigen-binding fragment thereof is a human or humanized antibody. In another embodiment, the antibody lacks an Fc portion. In yet another embodiment, the antibody is an F(ab′)₂, and Fab, or an Fv structure. In yet another embodiment, the antibody or antigen-binding fragment thereof is present in a pharmaceutically acceptable carrier. In a preferred embodiment, the inhibitor is an inhibitor of a protein kinase C signaling pathway (e.g., bisindolylmaleimide I, ruboxistaurin, or CGP41251). In another preferred embodiment, the inhibitor is an anti-VEGF antibody. In another preferred embodiment, the inhibitor is an Flk-1 antagonist (e.g., SU1498). In another embodiment, the neoplasm is a renal cell carcinoma, a lung cancer, a glioma, or a breast carcinoma.

In another aspect, the invention features a method of identifying a candidate compound that inhibits a neoplasm, the method involves contacting a cell that expresses an endocan nucleic acid molecule with a candidate compound, and comparing the level of expression of the nucleic acid molecule in the cell contacted by the candidate compound with the level of expression in a control cell not contacted by the candidate compound, where a decrease in expression of the endocan nucleic acid molecule identifies the candidate compound as a candidate compound that inhibits a neoplasm. In one embodiment, the decrease in expression is a decrease in transcription. In another embodiment, the decrease in expression is a decrease in translation. In one embodiment, the cell is in vivo. In a preferred embodiment, the cell in in vitro. In one embodiment, the cell is a human umbilical vein endothelial cell. In a preferred embodiment, the cell is grown in the presence of a tumor derived factor.

In another aspect, the invention features a method of identifying a candidate compound that inhibits a neoplasm, the method involves contacting a cell that expresses an endocan polypeptide with a candidate compound, and comparing the level of endocan polypeptide in the cell contacted by the candidate compound with the level of endocan polypeptide in a control cell not contacted by the candidate compound, where a decrease in the level of endocan polypeptide identifies the candidate compound as a candidate compound that inhibits a neoplasm. In one embodiment, the decrease in expression is assayed using an immunological assay, an enzymatic assay, or a radioimmunoassay. In another embodiment, the cell is a human umbilical vein endothelial cell. In a preferred embodiment, the cell is grown in the presence of a tumor derived factor. In another preferred embodiment, where the tumor-derived factor is present in conditioned culture media.

In another aspect, the invention features a method of identifying a candidate compound that inhibits a neoplasm, the method involves contacting a cell that expresses endocan with a candidate compound, and comparing endocan biological activity in the cell contacted by the candidate compound with the level of endocan biological activity in a control cell not contacted by the candidate compound, where a decrease in the level of endocan biological activity identifies the candidate compound as a candidate compound that inhibits a neoplasm. In one embodiment, the cell is a neoplastic cell. In another embodiment, endocan biological activity is assayed by measuring the viability or proliferation of the cell. In another embodiment, the cell is an endothelial cell. In another embodiment, the endothelial cell is a neoplasm endothelial cell. In another embodiment, the endocan biological activity is assayed by measuring tumor size, cell number, or viability. In yet another embodiment, cell is grown in the presence of a tumor-derived factor. In one preferred embodiment, the tumor-derived factor is present in conditioned culture media. In yet another embodiment, the endocan biological activity is promotion of angiogenesis.

In another aspect, the invention features a pharmaceutical composition containing an isolated endocan inhibitory nucleic acid molecule, or portion thereof, formulated in a pharmaceutically acceptable carrier. In one embodiment, the nucleic acid molecule is a double stranded nucleic acid molecule that decreases expression of an endocan nucleic acid molecule or polypeptide in a cell. In another embodiment, the nucleic acid molecule is an antisense nucleic acid molecule that decreases the expression of an endocan nucleic acid molecule or polypeptide in a cell.

In another aspect, the invention features a method of identifying a neoplasm endothelium-specific promoter. The method involves (a) providing an expression construct containing at least a tumor-factor responsive nucleic acid sequence and a minimal core promoter operably-linked to a nucleic acid sequence that encodes a protein; (b) transforming a cell with the construct; and (c) detecting expression of the construct in the cell, where a promoter that directs expression in the cell is a neoplasm endothelium-specific promoter. In one embodiment, the method further involves the steps of (d) providing an Hprt targeting vector containing the endothelium-specific promoter; (e) transforming a mouse with the in vector; and (f) detecting expression of the vector in neoplasm endothelium. In one embodiment, the minimal core promoter contains a portion of a Tie-2 promoter. In another embodiment, the encoded protein is a detectable reporter. In another embodiment, the tumor-factor responsive nucleic acid sequence is selected from the group consisting of tumor-responsive elements of an endocan promoter, tumor-responsive elements in promoters of transgenes that direct expression during tumor angiogenesis, and hypoxia response elements.

In another aspect, the invention features a method of delivering a polypeptide to a neoplasm. The method involves (a) providing a construct containing the promoter of the previous aspect; and (b) transforming a cell with the construct patient under conditions suitable for expressing the nucleic acid in the construct, whereby the construct expresses the product in the tumor.

In another aspect, the invention features a neoplasm endothelium-specific promoter containing at least a tumor-factor responsive nucleic acid sequence and a minimal core promoter, where the promoter when operably linked to a heterologous nucleic acid molecule directs expression of the nucleic acid molecule in a tumor endothelial cell. In one embodiment, the promoter contains at least a portion of an endocan or Flt-1 nucleic acid molecule.

In preferred embodiments of any of the above aspects, the patient sample is a blood sample or a tissue sample. In other preferred embodiments of any of the above aspects, the neoplasm is a renal cell carcinoma, a lung cancer, a glioma, or a breast carcinoma.

In other aspects, the invention features an expression construct containing the promoter of the previous aspect, where the promoter is positioned for expression and a transformed cell expressing this construct.

For the purpose of the present invention, the following abbreviations and terms are defined below.

By “administering” is meant the application, directly or indirectly, of a particular substance. For example, “administering an antisense RNA” would include administering antisense RNA or administering DNA that encodes an antisense RNA. No particular limitation on the form or manner of introducing the substance is implied by the use of the term “administering”.

By “antiangiogenic drugs” is meant chemicals that inhibit the growth, proliferation, maturation, or formation of new blood vessels.

By “anti-sense” is meant a nucleic acid sequence, regardless of length, that is complementary to the coding strand or mRNA of a nucleic acid sequence. Desirably the anti-sense nucleic acid is capable of decreasing the expression or biological activity of a nucleic acid or amino acid sequence. In a desirable embodiment, the decrease in expression or biological activity is at least 10%, relative to a control, more desirably 25%, and most desirably 50% or more. The anti-sense nucleic acid may contain a modified backbone, for example, phosphorothioate, phosphorodithioate, or other modified backbones known in the art, or may contain non-natural internucleoside linkages.

“Cell” as used herein may be a single-cellular organism, cell from a multi-cellular organism, or it may be a cell contained in a multi-cellular organism.

By “chemotherapeutic agent” is meant an agent that is used to kill cancer cells or to slow their growth. Accordingly, both cytotoxic and cytostatic agents are considered to be chemotherapeutic agents.

By “chemotherapy” is meant the treatment of a neoplasm.

By “chimeric antibody” is meant a peptide comprising at least the antigen-binding portion of an antibody molecule linked to at least a portion of another protein (typically an immunoglobulin constant domain).

By “derived from” is meant isolated from or having the sequence of a naturally occurring sequence (e.g., a cDNA, genomic DNA, synthetic, or combination thereof).

By “differentially expressed” is meant having a difference in the expression level of a nucleic acid or polypeptide. This difference may be either an increase or a decrease in expression, when compared to control conditions.

By “double stranded RNA” is meant a complementary pair of sense and antisense RNAs regardless of length. In one embodiment, these dsRNAs are introduced to an individual cell, tissue, organ, or to a whole animals. For example, they may be introduced systemically via the bloodstream. Desirably, the double stranded RNA is capable of decreasing the expression or biological activity of a nucleic acid or amino acid sequence. In one embodiment, the decrease in expression or biological activity is at least 10%, relative to a control, more desirably 25%, and most desirably 50%, 60%, 70%, 80%, 90%, or more.

By “duplex” is meant a domain containing paired sense and antisense nucleobase oligomeric strands. For example, a duplex comprising 29 nucleobases contains 29 nucleobases on each of the paired sense and antisense strands.

By “an effective amount” is meant the amount of a compound (e.g., a nucleobase oligomer) required to ameliorate the symptoms of a disease, inhibit the growth of a target cell, reduce the size or number of tumors, or inhibit the expression of a target gene, relative to the expression of the gene in an untreated patient. The effective amount of active compound(s) used to practice the present invention for therapeutic treatment of abnormal proliferation (i.e., cancer) varies depending upon the manner of administration, the age, body weight, and general health of the subject. Ultimately, the attending physician or veterinarian will decide the appropriate amount and dosage regimen. Such amount is referred to as an “effective” amount.

By “endocan nucleic acid” is meant a nucleic acid encoding a polypeptide substantially identical to the polypeptide defined by GenBank accession numbers CAA61597 (human endocan) or CAB60730 (mouse endocan). In one example, an endocan nucleic acid is defined by GenBank accession numbers X89426 (human endocan) and AJ249354 (mouse endocan).

By “endocan polypeptide” is meant a polypeptide having substantial identity to the polypeptide defined by GenBank accession numbers CAA61597 (human endocan) or CAB60730 (mouse endocan).

By “endocan biological activity” is meant promotion of neoplasm growth. For example, endocan may promote neoplasm growth by promoting or stabilizing the formation of neoplasm blood vessels.

By “tumor-factor responsive DNA element” is meant a cis-acting nucleic acid sequence that increases or decreases the expression of an operably linked nucleic acid in response to neoplasm-derived compound. A tumor-factor responsive DNA element increases or decreases such expression by at least 10%, 20%, 30%, or 40%, more preferably by at least 50%, 60%, or 70%, and most preferably by at least 75%, 80%, 90% or 95%. The modulation of expression may be the result of differential binding of protein factors on the DNA or a change in DNA conformation brought about by the environmental cue.

By “expression” is meant the detection of a gene product or protein product by standard art methods. For example, protein expression is often detected by immunological methods (e.g., western blotting); and DNA expression is often detected by Southern blotting, and RNA expression is detected by northern blotting or by RNAse protection assays.

By “Flt-1 nucleic acid” is meant a nucleic acid sequence substantially identical to Genbank Accession No: X51602

By “Flt-1 polypeptide” is meant a protein encoded by a Flt-1 nucleic acid molecule.

By “Flt-1 biological activity” is meant tyrosine kinase activity.

By “Flk-1 antagonist” is meant a substance that inhibits the biological activity of Flk-1 relative to a control. In some embodiments, a Flk-1 antagonist is SU1498, SU5416,

By “Flk-1 biological activity” is meant tyrosine kinase activity that mediates tumor progression associated with VEGF. “Flk-1 antagonists” may include anti-Flk-1 antibodies and Flk-1 fragments that block Flk-1 action.

By “high-level expression” is meant expression in a system of a particular RNA or protein that is increased by at least 10% or 25%, preferably by at least 30% or 50%, more preferably by at least 60%, 75%, 80%, 90%, or 100%, and most preferably by at least 200% (as caused by additional expression of the particular RNA or protein from a foreign construct introduced into the system), compared to a control system that did not receive the foreign construct. Thus, by “a promoter that directs high-level expression” is meant a promoter, which, when operably linked to a polynucleotide sequence in a construct which is then introduced into a cell, causes expression of the RNA or protein encoded by the polynucleotide sequence to increase by at least 25%, preferably by at least 50%, more preferably by at least 100%, and most preferably by at least 200%, compared to the expression of the same RNA or protein in a cell that did not receive the construct.

By “hybridize” is meant pair to form a double-stranded complex containing complementary paired nucleobase sequences, or portions thereof, under various conditions of stringency. (See, e.g., Wahl, G. M. And S. L. Berger (1987) Methods Enzymol. 152:399; Kimmel, A. R. (1987) Methods Enzymol. 152:507) For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York. Preferably, hybridization occurs under physiological conditions. Typically, complementary nucleobases hybridize via hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “immunological assay” is meant an assay that relies on an immunological reaction, for example, antibody binding to an antigen. Examples of immunological assays include ELISAs, Western blots, immunoprecipitations, and other assays known to the skilled artisan.

By an “inhibitory nucleobase oligomer” is meant a dsRNA, siRNA, shRNA, or mimetic thereof that inhibits the expression of a target gene. An inhibitory nucleobase oligomer typically reduces the amount of a target mRNA, or protein encoded by such mRNA, by at least 5%, more desirable by at least 10%, 25%, 50%, or even by 75%, 85%, or 90% relative to an untreated control. Methods for measuring both mRNA and protein levels are well known in the art; exemplary methods are described herein.

Preferably, an inhibitory nucleobase oligomer of the invention is capable of enhancing RNAi by decreasing mRNA or protein levels. Preferably a nucleobase oligomer of the invention includes from about 8 to 30 nucleobases. A nucleobase oligomer of the invention may also contain, for example, an additional 20, 40, 60, 85, 120, or more consecutive nucleobases that are complementary to a target polynucleotide of interest. The nucleobase oligomer (or a portion thereof) may contain a modified backbone. Phosphorothioate, phosphorodithioate, and other modified backbones are known in the art. The nucleobase oligomer may also contain one or more non-natural linkages.

By “isolated polynucleotide” is meant a nucleic acid (e.g., a DNA) that is free of the genes that, in the naturally occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “humanized antibody” is meant a peptide that comprises at least a portion of a human immunoglobulin. In one example, a humanized antibody comprises a framework region (FR) having substantial identity to the amino acid sequence of a human immunoglobulin and a complementarity determining region (CDR) having substantial identity to the amino acid sequence of a non-human immunoglobulin (the “import” sequences). By “complementarity determining region (CDR)” is meant the three hypervariable sequences in the variable regions within each of the immunoglobulin light and heavy chains. By “framework region (FR)” is meant the sequences of amino acids located on either side of the three hypervariable sequences (CDR) of the immunoglobulin light and heavy chains.

In general, a humanized antibody comprises a portion of at least one, and typically two, variable domains (Fab, Fab′, F(ab′)₂, Fabc, Fv) in which all or at least a portion of the CDR regions correspond to those of a non-human immunoglobulin and all or at least a portion of the FR regions are those of a human immunoglobulin consensus sequence. The humanized antibody optimally will comprise at least a portion of an immunoglobulin constant region (Fc), typically that of a human immunoglobulin. Typically, the antibody comprises both the light chain as well as at least the variable domain of a heavy chain. The antibody may also comprise the CH1, hinge, CH2, CH3, or CH4 regions of the heavy chain.

The FR and CDR regions of the humanized antibody may be mutagenized by substitution, insertion or deletion of at least 1, 2, 3, 4, 5, 6, 7, 9, or 10 residues, such that the CDR or FR residue at that site is not identical to a corresponding amino acid present at that position in either the consensus or the import antibody. Typically, at least 75%, preferably 90%, and most preferably at least 95% of the humanized antibody residues correspond to those of the FR or CDR sequence from which it is derived.

By “hypoxia response element” is meant a cis-acting element that increases or decreases expression of an operably linked nucleic acid sequence in response to hypoxic conditions (i.e., when dissolved oxygen (DO) concentrations are at least 5%, 10%, 20%, or 30%, more preferably at least 50% or 75%, or most preferably 90% or 100% below control conditions (e.g., 3.0 mg/l).

By “inhibitor of endocan biological activity” is meant a substance that decreases the biological activity of an endocan polypeptide contacted with the inhibitor relative to a corresponding control endocan polypeptide. In some examples, the decrease is by at least 5%, 10%, 20%, more preferably by at least 30%, 40%, 50%, 60%, 70%, or most preferably by at least 80%, 90%, or 100%. Inhibiting endocan biological activity may be accomplished at a variety of levels, and is understood to include but not to be limited to mechanisms that inhibit endocan gene transcription, endocan transcript processing and transport, translation of the endocan transcript, and post-translational modifications of the endocan polypeptide. Persons of skill in the art will appreciate that the extent of inhibition necessary to suppress growth of a tumor or neoplasm in a patient depends on a variety of factors including, but not limited to, the nature of the neoplasm and the patient's genetic and physiological constitution.

By “minimal core promoter” is meant a promoter that by itself is incapable of inducing endothelial-specific gene expression, but that induces endothelial cell-specific gene expression when coupled to an appropriate DNA sequence. Preferably, the minimal core promoter is a short 400-bp fragment of the Tie-2 gene promoter from −105 to +318 relative to transcriptional start site (GenBank accession number U53603).

By “monitoring” is meant the timely measurement of a particular parameter at intervals, the length of which depends on the parameter being measured and the effect that is being quantified. For example, monitoring levels of endocan RNA to determine how effective a particular regime of chemotherapy drug treatment is in suppressing growth of a neoplasm will require preferably determining the level of endocan RNA before treatment and then at least one time after treatment such that a practitioner could assess the effectiveness of the treatment.

By a “nucleobase oligomer” is meant any chain of nucleic acids or nucleic acid mimetics.

By a nucleobase oligomer that “reduces the expression” of a target gene is meant one that decreases the amount of a target mRNA, or protein encoded by such an mRNA, by at least about 5%, more desirable by at least about 10%, 25%, or even 50%, relative to an untreated control. Methods for measuring both mRNA and protein levels are well-known in the art; exemplary methods are described herein. Preferably a nucleobase oligomer of the invention includes from about 8 to 30 nucleobases. A nucleobase oligomer of the invention may also contain, for example, an additional 20, 40, 60, 85, 120, or more consecutive nucleobases that are complementary to an IAP polynucleotide. The nucleobase oligomer (or a portion thereof) may contain a modified backbone. Phosphorothioate, phosphorodithioate, and other modified backbones are known in the art. The nucleobase oligomer may also contain one or more non-natural linkages.

By “nucleic acid” is meant an oligomer or polymer of ribonucleic acid or deoxyribonucleic acid, for example, a dsRNA, siRNA, shRNA, or mimetic thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced cellular uptake and increased stability in the presence of nucleases.

Specific examples of some preferred modified nucleic acids or nucleobases envisioned for this invention may contain phosphorothioates, phosphotriesters, methyl phosphonates, short chain alkyl or cycloalkyl intersugar linkages or short chain heteroatomic or heterocyclic intersugar linkages. Most preferred are those with CH₂—NH—O—CH₂, CH₂—N(CH₃)—O—CH₂, CH₂—O—N(CH₃)—CH₂, CH₂—N(CH₃)—N(CH₃)—CH₂ and O—N(CH₃)—CH₂—CH₂ backbones (where phosphodiester is O—P—O—CH₂). Also preferred are oligonucleotides having morpholino backbone structures (Summerton, J. E. And Weller, D. D., U.S. Pat. No. 5,034,506). In other preferred embodiments, such as the protein-nucleic acid (PNA) backbone, the phosphodiester backbone of the oligonucleotide may be replaced with a polyamide backbone, the bases being bound directly or indirectly to the aza nitrogen atoms of the polyamide backbone (P. E. Nielsen, M. Egholm, R. H. Berg, O Buchardt, Science 199, 254, 1497). Other preferred oligonucleotides may contain alkyl and halogen-substituted sugar moieties comprising one of the following at the 2′ position: OH, SH, SCH₃, F, OCN, O(CH₂)ₙNH₂ or O(CH₂)ₙCH₃, where n is from 1 to about 10; C₁ to C₁₀ lower alkyl, substituted lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a conjugate; a reporter group; an intercalator; a group for improving the pharmacokinetic properties of an oligonucleotide; or a group for improving the pharmacodynamic properties of an oligonucleotide and other substituents having similar properties. Oligonucleotides may also have sugar mimetics such as cyclobutyls in place of the pentofuranosyl group.

Other preferred embodiments may include at least one modified base form. Some specific examples of such modified bases include 2-(amino)adenine, 2-(methylamino)adenine, 2-(imidazolylalkyl)adenine, 2-(aminoalklyamino)adenine, or other heterosubstituted alkyladenines. Each of the above is referred to as a “modification” herein.

By a nucleobase oligomer that “reduces the expression” of a target gene is meant one that decreases the amount of a target mRNA, or protein encoded by such mRNA, by at least about 5%, more desirable by at least about 10%, 25%, or even 50%, relative to an untreated control. Methods for measuring both mRNA and protein levels are well-known in the art; exemplary methods are described herein. Preferably, a nucleobase oligomer of the invention is capable of enhancing RNA interference.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

By “PCR” is meant the polymerase chain reaction well-known in the art as a method that is used to amplify in number a particular nucleic acid molecule. By “PCR” is meant to include methods that adapt the basic polymerase chain reaction to suit particular needs, such as RT-PCR (reverse transcriptase-PCR) designed to detect even very low levels of RNA. By “polypeptide” is meant any chain of amino acids, or analogs thereof, regardless of length or post-translational modification (for example, glycosylation or phosphorylation).

By “positioned for expression” is meant that the polynucleotide of the invention (e.g., a DNA molecule) is positioned adjacent to a DNA sequence that directs transcription and translation of the sequence (i.e., facilitates the production of, for example, a recombinant polypeptide of the invention, or an RNA molecule).

By “neoplasm” is meant a disease that is caused by or results in inappropriately high levels of cell division, inappropriately low levels of apoptosis, or both. For example, a neoplasm or cancer is an example of a proliferative disease. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

By “promoter” is meant a polynucleotide sufficient to direct transcription.

By “protein kinase C (PKC) signaling pathway” is meant a signal transduction pathway that contains protein kinase C as a signaling component. Inhibitors of protein kinase C which include bisindolylmaleimide I (BIM) are well-known in the art. Protein kinase C catalyzes the phosphorylation of tyrosine residues in certain proteins, including the VEGF receptor, which, when phosphorylated, is activated and leads to stimulation of angiogenesis by VEGF.

By “portion” is meant a fragment of a protein or nucleic acid that is substantially identical to a reference protein or nucleic acid, and retains at least 50% or 75%, more preferably 80%, 90%, or 95%, or even 99% of the biological activity of the reference protein or nucleic acid using a assay as described herein.

By “purified antibody” is meant an antibody that is at least 60%, by weight, free from proteins and naturally occurring organic molecules with which it is naturally associated. Preferably, the preparation is at least 75%, more preferably 90%, and most preferably at least 99%, by weight, antibody. A purified antibody of the invention may be obtained, for example, by affinity chromatography using a recombinant polypeptide of the invention and standard techniques.

By “radiation therapy” is meant the use of high-energy penetrating rays or subatomic particles to treat a neoplasm or a tumor. Types of radiation include x-ray, electron beam, alpha and beta particles, and gamma rays. Radioactive substances that emit high-energy rays used in radiation therapy include cobalt, radium, iridium, and cesium.

By “responsiveness of a neoplasm to therapy” is meant the extent to which a the characteristics of the neoplasm are altered in response to a treatment, relative to the characteristics of the neoplasm prior to treatment or relative to the characteristics of a corresponding untreated control neoplasm. Responsiveness of a neoplasm to therapy may be measured by assaying the size, growth rate, nutrient uptake, gene expression, or peptide expression of the neoplasm. In one example, peptide expression is monitored by assay the amount of a factor secreted by the neoplasm. Those of skill in the art will appreciate that the magnitude of the change in the character of the neoplasm or the biological system, is used as indicia of the responsiveness of the neoplasm to therapy.

By “reporter gene” is meant a gene encoding a polypeptide whose expression may be assayed; such polypeptides include, without limitation, —glucuronidase (GUS), luciferase, chloramphenicol transacetylase (CAT), and beta-galactosidase.

By “specifically binds” is meant a compound or antibody which recognizes and binds a polypeptide of the invention but which does not substantially recognize and bind other molecules in a sample, for example, a biological sample, which naturally includes a polypeptide of the invention.

By “shRNA” is meant an RNA comprising a duplex region complementary to an mRNA. For example, a short hairpin RNA (shRNA) may comprise a duplex region containing nucleoside bases, where the duplex is between 17 and 29 bases in length, and the strands are separated by a single-stranded 4, 5, 6, 7, 8, 9, or 10 base linker region. Optimally, the linker region is 6 bases in length.

By “siRNA” is meant a double stranded RNA comprising a region of an mRNA. Optimally, an siRNA is 17, 18, 19, 20, 21, 22, 23, or 24 nucleotides in length and has a 2 base overhang at its 3′ end. siRNAs can be introduced to an individual cell, tissue, organ, or to a whole animals. For example, they may be introduced systemically via the bloodstream. Such siRNAs are used to downregulate mRNA levels or promoter activity. Desirably, the siRNA is capable of decreasing the expression or biological activity of a nucleic acid or amino acid sequence. In one embodiment, the decrease in expression or biological activity is at least 10%, relative to a control, more desirably 25%, and most desirably 50%, 60%, 70%, 80%, 90%, or more. The siRNA may contain a modified backbone, for example, phosphorothioate, phosphorodithioate, or other modified backbones known in the art, or may contain non-natural internucleoside linkages. Such siRNAs are used to downregulate mRNA levels or promoter activity.

By “specific activity of the endocan polypeptide” is meant the amount of endocan protein activity present in a sample or a system, compared to the amount of total protein present in the same sample or system. This is a measure of the concentration of functional endocan protein present in a sample or system. Methods to determine endocan activity levels are well-known in the art.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and most preferably 90% or even 95% identical at the amino acid level or nucleic acid level to the sequence used for comparison. The comparison is over at least 25-50 nucleotides, more preferably 50-100 or 100-200 nucleotides, and most preferably 200-400, 400-600, 600-800, or even 800-1000 nucleotides.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “targets a gene” means specifically binds to and decreases the expression of the gene. For example, an inhibitory nucleic acid binds to and decreases the expression of a complementary target gene. Such a decrease is by at least 10%, 25%, 50%, 75%, or 100% relative to the expression of a corresponding control gene.

By “transformed cell” is meant a cell into which (or into an ancestor of which) has been introduced, by means of recombinant DNA techniques, a polynucleotide molecule encoding (as used herein) a polypeptide of the invention.

By “transgene” is meant any piece of DNA that is inserted by artifice into a cell and becomes part of the genome of the organism that develops from that cell or, in the case of a nematode transgene, becomes part of a heritable extrachromosomal array. Such a transgene may include a gene which is partly or entirely heterologous (i.e., foreign) to the transgenic organism, or may represent a gene homologous to an endogenous gene of the organism.

By “transgenic” is meant any cell which includes a DNA sequence which is inserted by artifice into a cell and becomes part of the genome of the organism which develops from that cell or part of a heritable extrachromasomal array. As used herein, the transgenic organisms are generally transgenic rodents, such as mice and rats.

By “therapy” is meant administering a pharmaceutical composition for therapeutic purposes. It refers to administering treatment to a patient already suffering from a disease to improve the patient's condition.

By “neoplasm” is meant an abnormal mass of cells that arises from cells of normal tissue through uncontrolled cell division, and that grows abnormally in rate and/or structure.

By “tumor-responsive element” is meant a cis-acting element that stimulates or suppresses expression of an operably linked nucleic acid sequence in response to a factor secreted by a neoplasm.

By “vascular endothelial growth factor (VEGF)” is meant a mammalian growth factor that is homologous to a growth factor as defined in U.S. Pat. Nos. 5,332,671; 5,240,848; 5,194,596; or Charnock-Jones et al. (Biol. Reproduction 48: 1120-1128, 1993), and having VEGF biological activity. VEGF exists as a glycosylated homodimer and includes at least four different alternatively spliced isoforms. Although human VEGF is preferred, the invention is not limited to human forms and can include other animal forms of VEGF (e.g. mouse, rat, dog, or chicken).

By “VEGF biological activity” is meant enhancing or supporting the growth of vascular endothelial cells or umbilical vein endothelial cells or inducing angiogenesis. As used herein, VEGF includes any VEGF family member or isoform (e.g. VEGF-A, VEGF-B, VEGF-C, VEGF-D, or VEGF-E).

By “vector” is meant a DNA molecule, usually derived from a plasmid or bacteriophage, into which fragments of DNA may be inserted or cloned. A recombinant vector will contain one or more unique restriction sites, and may be capable of autonomous replication in a defined host or vehicle organism such that the cloned sequence is reproducible. A vector contains a promoter operably linked to a gene or coding region such that, upon transfection into a recipient cell, an RNA is expressed.

Other features and advantages of the invention will be apparent from the following description of the preferred embodiments thereof, and from the claims.

### BRIEF DESCRIPTION OF THE DRAWINGS

FIGS. 1A-1D show endocan mRNA expression in tissues of adult mice. FIG. 1A is a Northern blot showing endocan expression in the brain, kidney, liver, lung, skeletal muscle (Sk M), spleen and heart of an adult mouse. These results are representative of two independent experiments. FIGS. 1B-1D show In situ hybridization assays of endocan and vWF expression in adult mouse tissues. The magnification used is 20× (FIGS. 1B, 1C) and 40× (FIG. 1D).

FIGS. 2A-2E show endocan mRNA expression in tumors and embryos. (FIG. 2A is a Northern blot showing endocan expression in human renal cell carcinoma (RCC), human non-small lung cancer (HLCC), rat glioma (C6) xenografts, and in mouse breast carcinoma from MMTV-c-neu transgenic mice (B. Ca). (These abbreviations are used throughout the figures.) The results of experiments from three independent animals are shown in FIGS. 2B and 2C. FIGS. 2B and 2C show in situ hybridizations of endocan and vWF in xenografts or in mouse breast carcinoma. In HLCC (FIG. 2C), the left half of the field represents normal mouse subcutaneous tissue.

FIG. 2D is a Northern blot showing endocan, vWF, and VEGF expression in mouse embryos between E4.5 and E18.5. FIG. 2E shows in situ hybridization of endocan and vWF in an E12.5 mouse embryo. FIGS. 2E-A and 2E-D, 2E-B and 2E-E, 2E-C and 2E-F are serial sections. In FIG. 2E-C, asterisks indicate the lumen of the dorsal aorta. Sections are shown at magnifications of 20× (FIG. 2B) and 40× (FIGS. 2C, 2D).

FIGS. 3A-3J show endocan mRNA expression in serum-starved human umbilical vein endothelial cells (HUVECs) treated with tumor cell-conditioned medium or growth factors. In FIG. 3A, HUVECs were treated in the absence (C) or presence of conditioned medium from human renal cell carcinoma (RCC-CM), human lung cell carcinoma (HLCC-CM), or rat glioma (C6-CM) cultures. In FIG. 3B, HUVECs were incubated with conditioned medium from human renal cell carcinoma for 3 hours (3 h), 6 hours, 12 hours, or 24 hours. In FIG. 3C, HUVECs were treated in the absence (C) or presence of conditioned medium from human renal cell carcinoma (R) with or without the addition of antibodies to VEGF (aV) or EGF (aE). In FIG. 3D, HUVECs were treated in the absence (C) or presence of VEGF (V), EGF (E) with or without the addition of antibodies against VEGF (aV) or EGF (aE). In FIG. 3E, HUVECs were treated in the absence (C) or presence of increasing doses of VEGF (ng/ml). In FIG. 3F, HUVECs were treated in the absence (C) or presence of VEGF for the times indicated. In FIG. 3G, HUVECs were treated in the absence (C) or presence of VEGF (V) with or without bisindolylmaleimide I (BIM), PD98059 (PD), an inhibitor of MAP kinase kinase (MEK), or LY294002 (LY), a specific phosphatidylinositol 3-kinase (Pl 3-kinase) inhibitor. In FIG. 3H, HUVECs were treated in the absence (C) or presence of conditioned medium from human renal cell carcinoma with or without BIM, PD98059 (PD), or LY294002 (LY). In FIG. 3I, HUVECs were treated in the absence (C) or presence of either VEGF (V), placenta growth factor (PlGF) or phorbol 13-myristate 12-acetate (PMA) with or without SU1498 (SU), a Flk-1 antagonist. In FIG. 3J, endocan expression was assayed in serum-starved HUVECs estimated to be 50%, 80% or 100% confluent, as indicated. All results are representative of three independent experiments.

FIG. 4 shows endocan mRNA expression in serum-starved HUVECs treated with primary cell-conditioned medium or EGF. HUVECs were treated in the absence (C) or presence of conditioned media from cultured primary human keratinocytes (K-CM) and human renal tubule epithelial cells (RT-CM) with or without anti-EGF antibody (aE). The results are representative of three independent experiments.

FIG. 5 is a table (TABLE 1) that summarizes the expression patterns observed for the indicated Hprt-targeted promoters.

FIGS. 6A-6D are micrographs showing Flt-1 and vWF promoter activity in tumor xenografts. Uniform and strong LacZ staining was detected in Lewis lung carcinoma (FIG. 6A) and B16-F1 melanoma (FIG. 6B) xenografts from Flt-1-lacZ-Hprt mice. In contrast, there was no detectable reporter gene activity in Lewis lung carcinoma (FIG. 6C) and B16-F1 melanoma (FIG. 6D) xenografts from vWF-lacZ-Hprt mice. Arrowheads indicate the X-Gal reaction product in capillaries of adjacent skeletal muscle. Broken lines indicate the border between the tumor layer and the adjacent skeletal muscle layer. Micrographs of the tissue are shown at 100× optical magnification. The results are representative of at least two independent experiments.

FIG. 7A is an RNAse protection assays showing that conditioned medium from tumor cells induced Flt-1, but not vWF, mRNA and promoter activity. In FIG. 7A, HUVECs were serum-starved and then incubated with or without tumor conditioned medium. In RNase protection assays, a [α-³²P] UTP-labeled 413-bp human Flt-1 antisense riboprobe was incubated with 10 μg of yeast RNA (lane 1) or total RNA from untreated HUVECs (lane 2), HUVECs treated with conditioned medium from Lewis lung carcinoma (LLC) cells (lane 3), or HUVECs treated with conditioned medium from B16-F1 melanoma cells (lane 4). A [α-³²P] UTP-labeled 392-bp human vWF antisense riboprobe was incubated with 10 μg of yeast RNA (lane 5) or total RNA from untreated HUVECs (lane 6), HUVECs treated with conditioned medium from Lewis lung carcinoma (LLC) cells (lane 7), or HUVECs treated with conditioned medium from B16-F1 melanoma cells (lane 8). The protected fragment (316-bp hFlt-1 and 288-bp hvWF) represents the human Flt-1 and vWF transcripts, respectively. A [α-³²P] UTP-labeled human GAPDH antisense riboprobe was hybridized with total RNA as an internal control. Densitometry was used to calculate the ratio of Flt-1, vWF and GAPDH signals. The results are representative of two independent experiments.

FIG. 7B is a graph showing relative promoter activity in HUVECs transiently transfected with Flt-1-luc and vWF-luc-2 then incubated with Lewis lung carcinoma or B16-F1 melanoma conditioned medium for 24 hours. The results show the mean and standard deviations of luciferase light units (relative to untreated cells) obtained in triplicate from three independent experiments.

### DETAILED DESCRIPTION OF THE INVENTION

We have found that endocan is predominantly expressed within the endothelial lining of tumor vessels. The data described herein also indicate that endocan interacts with neighboring tumor cells to induce cell proliferation. Accordingly, the invention provides a number of important advances and advantages for monitoring and suppressing tumors or cancerous growths for which there otherwise remains a dearth of therapeutic treatments. The use of endocan as a marker for monitoring the responsiveness of a tumor to therapy, and the strategy of inhibiting endocan biological activity to suppress a tumor provide additional tools with which to combat these abnormal growths.

There are several aspects of this study that are novel and unexpected, and which have significant biological implications. First, we believe our results with endocan provide the only example of an endothelial cell marker that is expressed in the vasculature of tumors, but not during embryonic angiogenesis. Second, our results suggest that the limited expression of endocan in normal mouse tissues is mediated by active repression of the endocan gene via tissue-derived soluble factors. Our findings provide a unique model in which tumor cells induce tumor blood vessel endothelium to express endocan by a VEGF-dependent pathway. Once secreted, endocan may then act in a paracrine manner to stimulate tumor growth. These data have important implications from a mechanistic, diagnostic, and therapeutic standpoint. Based on our model, endocan is believed to be a potential therapeutic target for inhibiting tumor growth in humans.

Studies described in this specification are also the first investigations to demonstrate tumor endothelial cell-specific differences in promoter function (Flt vs. Tie-2, vWF). The studies described below elucidate transcriptional control mechanisms that are specific to tumor endothelium. Accordingly, the invention features therapeutic nucleic acid and protein compositions and methods of delivering such compositions. In one example, therapeutic modules are packaged into viral vectors, and administered systemically. Alternatively, they may be linked to target genes and delivered locally to a neoplasm. Alternatively, therapeutic nucleic acids are transfected into bone marrow-derived progenitor cells ex-vivo and the resulting clones administered to patients, with the anticipation that the genetically engineered cells will contribute to the endothelial cell lining of tumor blood vessels.

### Methods

Materials

Recombinant human VEGF₁₆₅, EGF, basic fibroblast growth factor (bFGF), platelet-derived growth factor (PDGF)-BB and placenta growth factor (PIGF) were purchased from R&D Systems (Minneapolis, Minn.). Neutralizing antibodies to VEGF, EGF, bFGF, and PDGF were also obtained from R&D Systems. Bisindolylmaleimide I (BIM), LY294002 (specific phosphatidylinositol 3-kinase (Pl 3-kinase) inhibitor), PD98059 (inhibitor of MAP kinase kinase (MEK)), and SU1498 (a selective inhibitor of Flk-1 kinase (IC₅₀=700 nM)), were obtained from Calbiochem (La Jolla, Calif.). Phorbol 13-myristate 12-acetate (PMA) was from Sigma (St. Louis, Mo.).

Cell Culture

Human non-small lung cancer cells (NCI-H1437, ATCC (American Type Culture Collection) CRL-5872) and rat glioma cells (C6, ATCC CCL-107) were grown in DMEM (Life Technologies Inc., Gaithersburg, Md.) containing 10% FBS (Life Technologies Inc.). Human renal cell carcinoma cells (786-0, ATCC CRL-1932) were maintained in RPMI 1640 (Life Technologies Inc.) containing 10% FBS. HUVECs, human epidermal keratinocytes and human renal tubule epithelial cells were obtained from Clonetics (San Diego, Calif.) and grown in EGM-2-MV, KGM and REGM, respectively (Clonetics). All cells were maintained at 37° C. under 5% CO₂ in humidified air. Endothelial cells were grown to 90% confluence, serum-starved overnight in EBM-2 medium containing 0.5% FBS and incubated in the absence or presence of tumor cell-conditioned medium or growth factors at the doses and for the times indicated. Unless otherwise stated, endothelial cells were incubated with 20 ng/ml VEGF for 6 hours. In inhibition studies, serum-starved endothelial cells were preincubated with 5 μM BIM, 20 μM LY294002, 50 μM PD98059, or 20 μM SU1498 for 30 minutes before a 6 hour treatment with tumor conditional medium or VEGF. For neutralization studies, tumor conditional medium or growth factors were preincubated with antibodies for 1 hour at 37° C. and the mixture was then added to serum-starved HUVECs.

RNA Probe Preparation and In Situ Hybridization

Mouse endocan cDNA (500 base pair, nucleotides 64-563, GenBank accession AJ249354) was prepared by reverse transcriptase polymerase chain reaction (RT-PCR) of mouse kidney RNA and subcloned into the pBluescript vector (Stratagene, La Jolla, Calif.) to generate endocan-pBlue. A mouse vWF cDNA IMAGE clone (IMAGE 1055065) was purchased from Research Genetics (Huntsville, Ala.). The sequences were confirmed by automated DNA sequencing. Non-radioactive in situ hybridization was performed as previously described (Berger et al., J. Comp. Neurol. 433:101-114, 2001). Briefly, endocan-pBlue was linearized with HindIII or NotI, and sense and antisense digoxigenin (DIG)-labeled cRNA probes were generated with the DIG RNA labeling kit (Roche, Indianapolis, Ind.). Ten-μm frozen sections were collected by cryostat and captured onto Superfrost plus microscope slides (Fisher Scientific, Pittsburgh, Pa.). The sections were then fixed and acetylated, and hybridized at 68° C. over three nights to the sense or antisense endocan probes (approximate concentration 100 ng/ml). Hybridized probe was visualized using alkaline phosphatase-conjugated anti-DIG Fab fragments (Roche) and 5-Bromo-4-chloro-3-indolyl-phosphate/Nitro blue tetrazolium (BCIP/NBT) substrate (Kierkegard and Perry Laboratories, Gaithersburg, Md.). Tissue sections were rinsed several times in 100 mM Tris, 150 mM NaCl, 20 mM EDTA pH 9.5, and mounted with glycerol gelatin (Sigma).

In Vivo Tumor Models

Cultured human non-small lung cancer cells (NCI-H1437), rat glioma cells (C6) and human renal cell carcinoma cells (786-0) were grown to confluence, trypsinized, and washed with phosphate buffered saline (PBS). A total of 5 million cells were suspended in 200 μl PBS, and the resulting suspension was injected subcutaneously into the right flank of 7- to 9-week-old athymic nude mice. The tumors were allowed to grow to 0.25-0.5 cm³, at which time the mice were sacrificed and tumor tissues were harvested for in situ hybridization and Northern blot assays. The breast cancer tumor tissues were obtained from MMTV-c-neu transgenic mice (Charles River Labs, Wilmington, Mass.).

RNA Preparation and Northern Blot

Cultured cells and tissues were harvested for total RNA extraction, using the Trizol reagent (Life Technologies). Five μg total RNA from cells or 10 μg RNA from tissues were loaded on a 0.7% formaldehyde-containing agarose gel. The RNA was transferred to nylon membrane, covalently cross-linked by UV radiation, prehybridized for 6 hours, and hybridized for 18 hours at 42° C. with 10⁶ cpm/ml [³²P] dCTP-labeled human endocan, mouse endocan, mouse vWF, or mouse VEGF cDNA probe.

Mouse Embryo Analysis

Mouse embryos (E12.5) were isolated, frozen, and cryostat sectioned for in situ hybridization studies. A mouse embryo full stage blot was purchased from Seegene Inc. (Seoul, Korea) and used for the Northern analysis.

### EXAMPLES

### Endocan is not Widely Expressed in the Normal Endothelium of the Adult Mouse

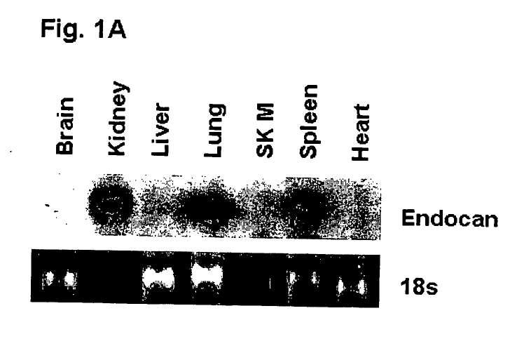

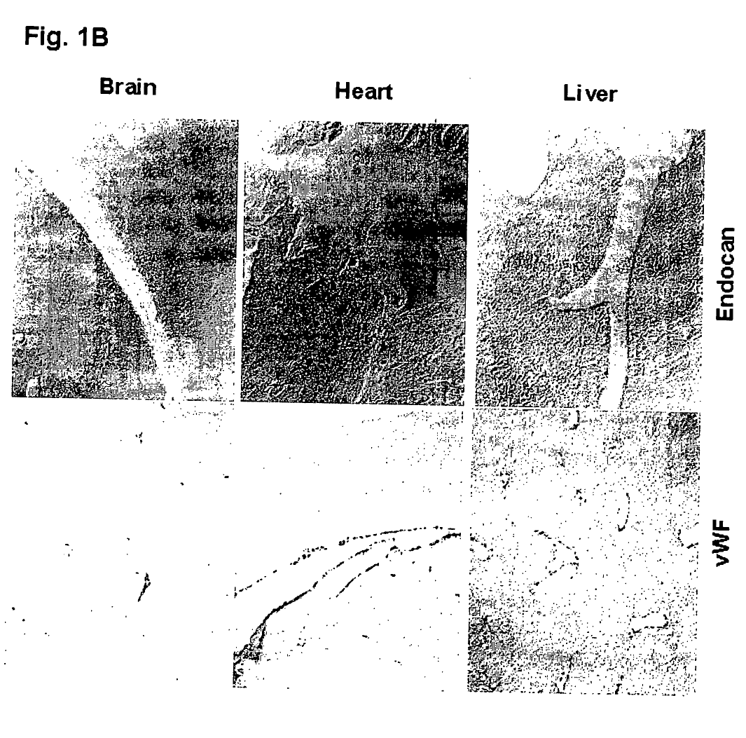

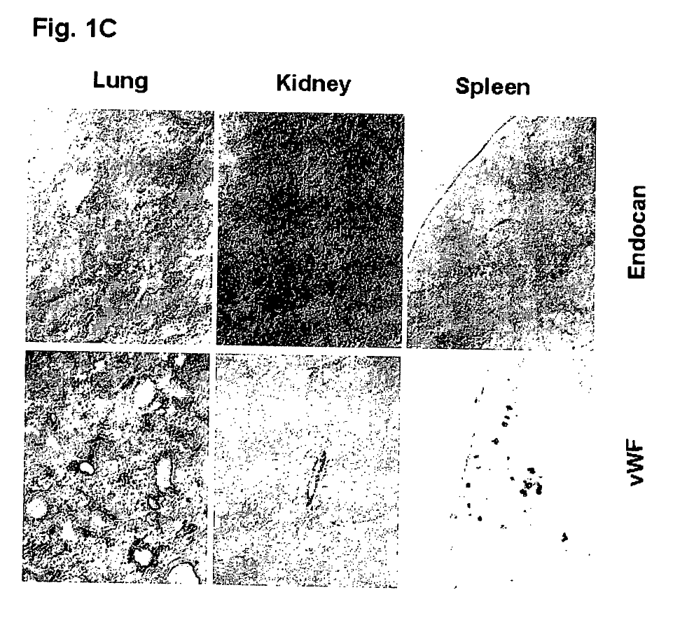

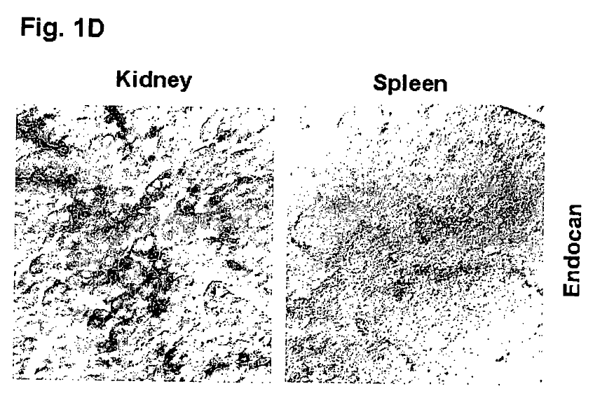

In Northern blot assays, expression of the endocan gene in mice was detected in the kidney, lung and spleen, but not the brain, liver, skeletal muscle, or heart (FIG. 1A). In in situ hybridization studies, a positive signal was detected in the kidney and spleen, but not in the other organs (FIGS. 1B-1D). In the kidney, endocan transcripts were localized to peritubular cells of the medulla and cortex (FIG. 1D). In the spleen, endocan expression was observed in a subpopulation of mononuclear cells surrounding the white pulp (FIG. 1D). Despite the positive signal in Northern blot assays, endocan expression was undetectable in repeated in situ hybridizations of the adult mouse lung (FIG. 1C). This discrepancy suggests that endocan is widely expressed in one or more cell types in the lung, at levels below the limit of detection of the in situ hybridization assay. To compare the distribution of endocan with a commonly employed marker of endothelial cells, we carried out in situ hybridizations with an antisense von Willebrand factor (vWF) probe. In these studies, vWF was detected in a subset of endothelial cells in virtually every tissue examined (FIGS. 1B, 1C). Taken together, these findings suggest that the endocan gene is not widely expressed in the normal endothelium of the adult mouse.

### Endocan is Expressed in the Endothelium of Tumor Vascular Beds

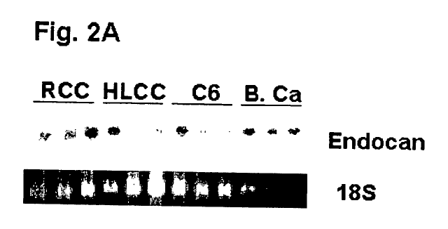

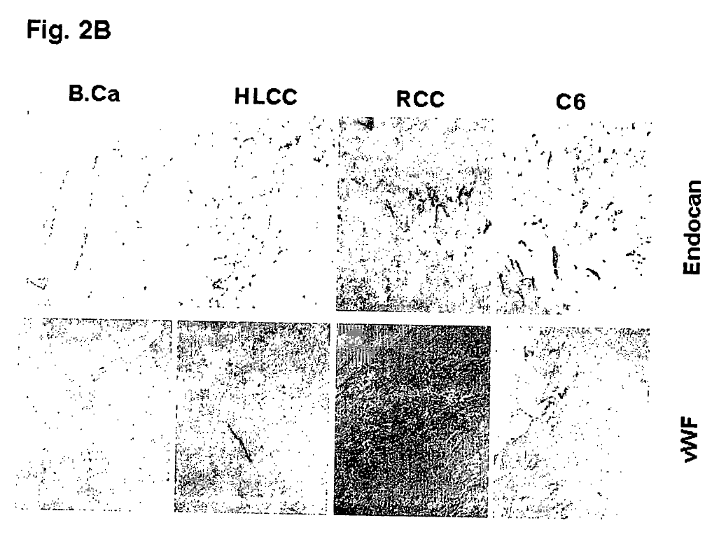

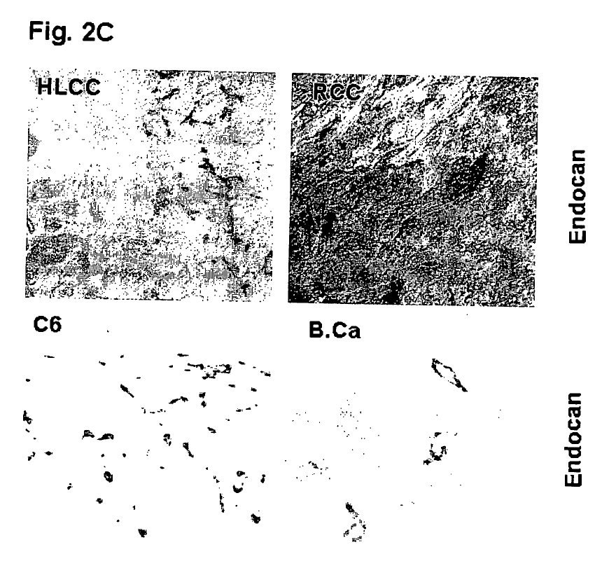

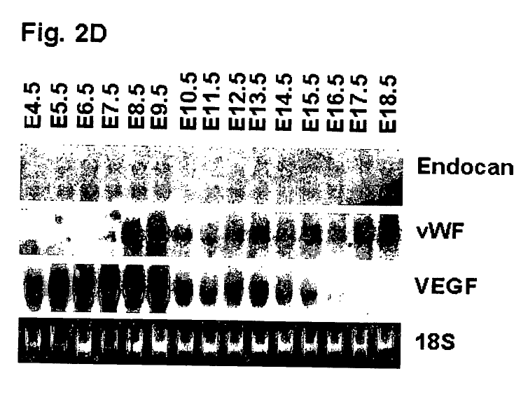

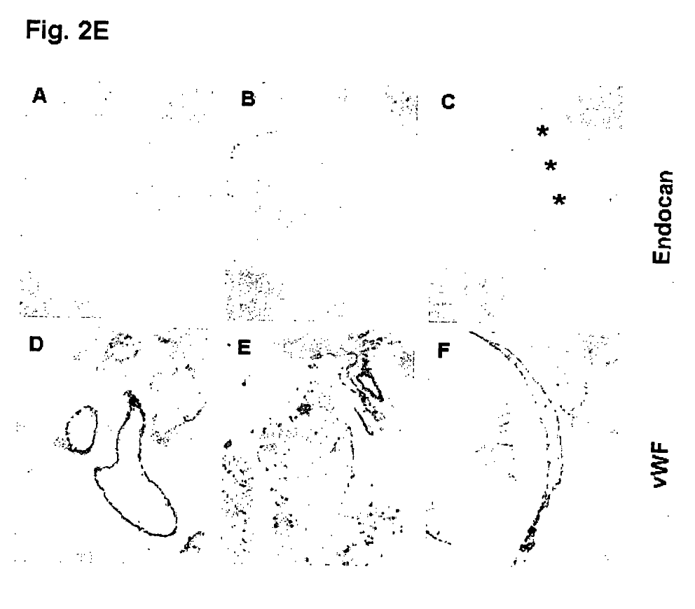

Endocan transcripts were detected in Northern blot analyses of tumor xenografts (human renal cell carcinoma, human non-small lung cancer and rat glioma) and mouse breast carcinoma from MMTV-c-neu transgenic mice (FIG. 2A). In contrast, there was no detectable expression of endocan in Northern blot assays of cultured human renal cell carcinoma, human non-small lung cancer and rat glioma cells. In in situ hybridization studies, endocan transcripts in the tumors were localized to the endothelium (FIGS. 2B, 2C). Expression of vWF, which is a commonly used marker of tumor endothelial cells, was expressed at much lower levels within the tumor vasculature (FIG. 2B). These data suggest that endocan is preferentially expressed in tumor endothelium.

### Endocan is not Expressed During Embryogenesis

In Northern blot analyses of mouse embryos, endocan mRNA was undetectable between embryonic stages E4.5 and E18.5 (FIG. 2D). In contrast, vWF was expressed at high levels throughout later stages of development. The embryonic expression of endocan and vWF was confirmed using in situ hybridizations. In E12.5 embryos, endocan expression was undetectable, while vWF was expressed in the endotbelial lining of most blood vessels (FIG. 2E). We conclude that the endocan gene is expressed during tumor angiogenesis, but not during normal embryonic development.

### Endocan Expression in Cultured HUVECs is Upregulated by Tumor Cell-Conditioned Medium

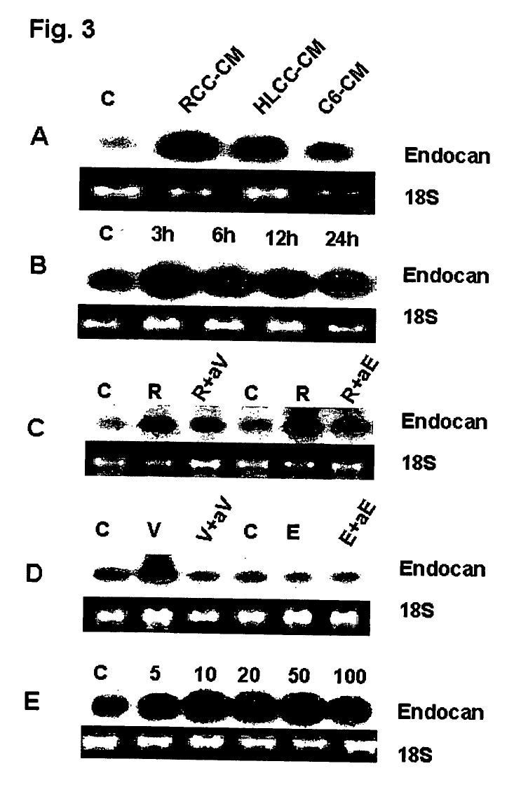

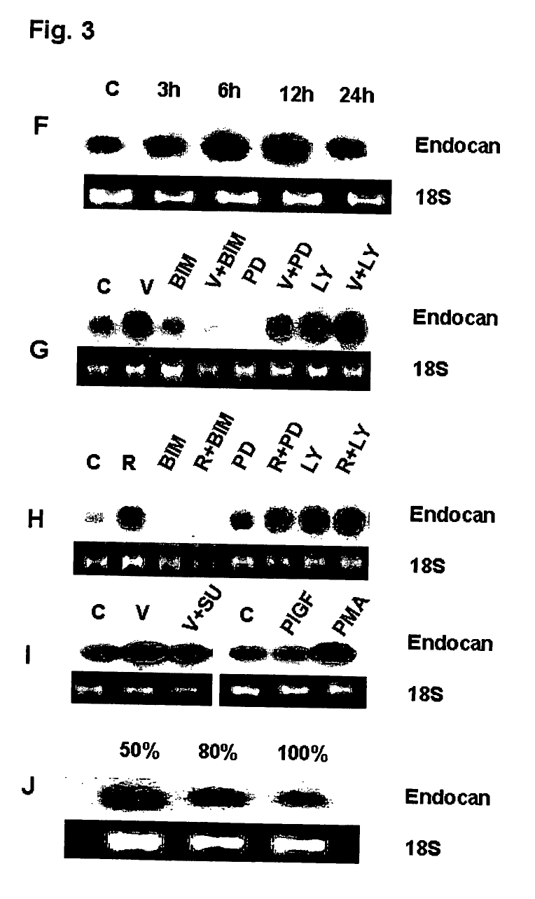

The addition of conditioned medium from renal cell carcinoma cells, human non-small lung cancer cells, or rat glioma cells resulted in a significant upregulation of endocan mRNA in HUVECs (FIG. 3A). In time course studies, conditioned medium from renal cell carcinoma cells resulted in maximal induction of endocan expression at 3 hours (FIG. 3B). The tumor cell-mediated induction of endocan was abrogated by pre-incubation with anti-VEGF antibody, but not by antibodies against EGF, basic fibroblast growth factor (bFGF), or platelet-derived growth factor (PDGF) (FIG. 3C shows anti-VEGF and anti-EGF). These results suggested that tumor-mediated induction of endocan in endothelial cells was mediated, at least in part, by VEGF. Consistent with these findings, the addition of VEGF to HUVECs resulted in a time and dose dependent increase of endocan mRNA (FIGS. 3D-3F). To determine the relative roles of PKC, ERK1/2 MAPK and PI3K signaling pathways in mediating the response to tumor cell-conditioned medium or VEGF, cells were pre-treated with BIM, an inhibitor of protein kinase C, PD90985, an inhibitor of MAP kinase kinase (MEK), and LY294002, a specific phosphatidylinositol 3-kinase (Pl 3-kinase) inhibitor. Endocan induction by either tumor cell-conditioned medium or VEGF was completely inhibited by the addition of BIM, but not by PD90985 or LY294002, implicating a role for the PKC signaling pathway (FIGS. 3G, 3H) in endocan induction. Preincubation of HUVECs with the Flk-1 antagonist, SU1498 blocked VEGF-mediated induction to endocan, whereas the addition of placenta growth factor (PlGF) to HUVECs did not alter endocan expression levels (FIG. 3I). These latter results suggest that VEGF stimulation of endocan is mediated by the Flk-1 receptor. To test whether VEGF was sufficient for endocan expression in vivo, we probed the mouse embryo blot with a mouse VEGF cDNA probe. In contrast to the distinct absence of endocan, VEGF mRNA was detected at all embryonic stages (FIG. 2D). These results suggest that VEGF is not sufficient for mediating expression of endocan during normal development. Finally, to test whether endocan expression was influenced by the proliferative state of endothelial cells, we carried out Northern blot analyses of endocan in HUVECs plated and harvested at different densities. As shown in FIG. 3J, endocan expression was inversely correlated with cell density.

### Endocan Expression in Cultured HUVECs is Downregulated by Conditioned Media from Primary Dermal Keratinocytes and Renal Proximal Tubule Epithelial Cells

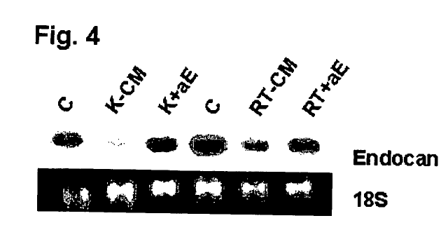

We have previously shown that endocan is expressed in many types of primary human endothelial cells, including HUVECs, coronary artery endothelial cells, pulmonary artery endothelial cells and dermal microvascular endothelial cells. The discrepancy between the pan endothelial cell expression in vitro and the tumor-restricted expression in vivo suggests that the endocan gene is de-repressed when endothelial cells are uncoupled from their native environment. To test this hypothesis in the in vitro setting, we measured endocan mRNA in response to conditioned medium from human primary non-transformed cells, including keratinocytes and renal tubule epithelial cells. Endothelial expression of endocan was downregulated under these conditions (FIG. 4). Pre-incubation with anti-EGF antibody prevented the response, invoking a role for this growth factor in repressing endocan expression in normal tissues (FIG. 4).

### The Flt-1 Promoter Contains Information for Expression in Tumor Endothelium

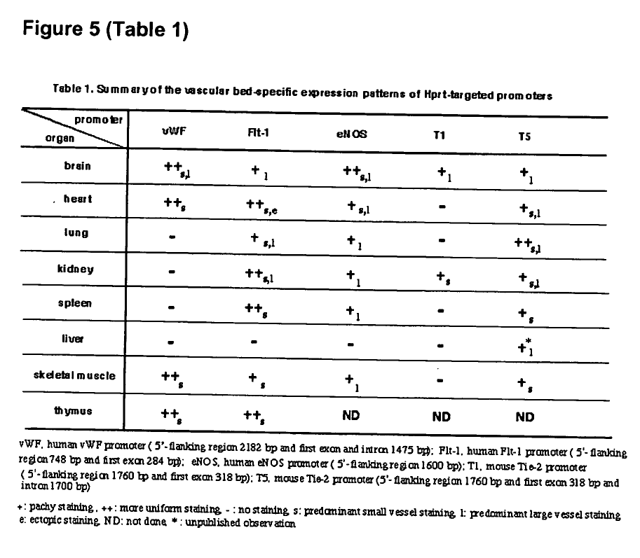

To control for the effects of copy number and integration site on promoter activity in transgenic mice, homologous recombination was employed to target single copy transgenes to a defined locus of the mouse genome. Using this approach, over 12 different endothelial cell gene promoters have been successfully targeted to the Hprt locus of mice (a subset of these results is presented in FIG. 5 (Table 1)). Interestingly, each of the promoters directs expression in a unique subset of endothelial cells or blood vessel types.

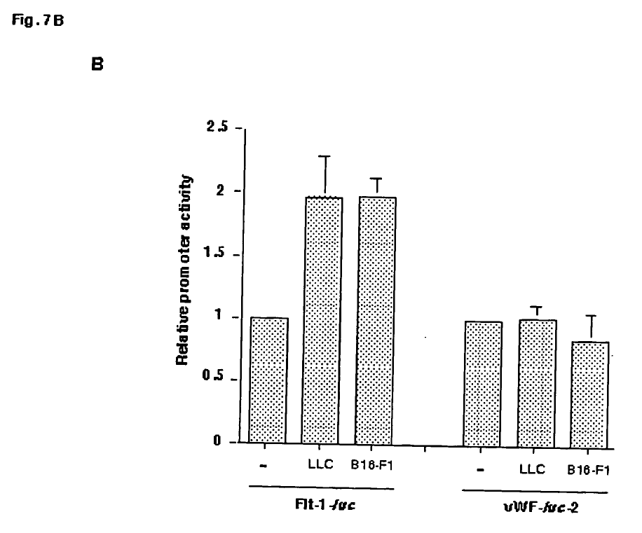

Most importantly, the promoter of the Flt-1 gene 748 and +284, relative to transcriptional start site (base #1196 in accession D64016=+1), but not the Tie-2 or vWF genes, was shown to direct expression in the endothelium of tumor xenografts (Lewis Lung Carcinoma and B16-F1 melanoma). These results were recapitulated in the tissue culture dish; conditioned medium from tumor cells resulted in a significant upregulation of Flt-1 mRNA (Genbank Accession No: X51602) and promoter activity, but no change in vWF levels (see FIGS. 7A and 7B). Taken together, these results suggest that: 1) Hprt locus targeting is a valuable tool for studying vascular bed-specific gene regulation; 2) different endothelial cell promoters are regulated by distinct transcriptional mechanisms in the intact endothelium; and 3) tumor angiogenesis results in the differential activation of endothelial cell-specific promoters. It follows from these observations that the co-culture and Hprt locus targeting systems will provide complementary approaches to rapidly map the tumor-responsive transcriptional control elements in the Flt-1 promoter.

### Tumor Endothelial Cell-Specific Regulation of the Endocan Gene

The 5′ flanking region (3888 base pairs) of the human endocan gene has been cloned and sequenced (Tsai et al., J. Vasc. Res. 39:148-159, 2002) (GenBank accession number AY054987, −3888, relative to start site of transcription, where base pair 3888 is +1 (i.e., the start site of transcription)). The full-length promoter directed high-level expression of the luciferase reporter gene in cultured endothelial cells, but not in non-endothelial cell types. In 5′ deletion analyses, a region spanning −81-+58 (relative to transcriptional start site which is designated position +1) was shown to contain information for endothelial cell-specific expression. In the next set of experiments, the endothelial cell-specific promoter is coupled to LacZ in the Hprt targeting vector (Hprt targeting vector originally from Bronson et al. Proc Natl Acad Sci USA. 93: 9067-72, 1996). The resulting construct is then used to generate Hprt-targeted animals. The tissues from these mice are analyzed for transgene expression. Based on the expression pattern of the endogenous endocan gene, as described herein, the reporter gene will likely be specifically activated in tumor endothelium. Transient transfection assays in primary human endothelial cells are employed to test the response of the full length promoter to conditioned medium from various tumor cell lines. Internal deletions and mutations are introduced to define the conditioned-medium-inducible cis-regulatory elements. The results will be confirmed by targeting similar mutants to the Hprt locus of mice.

### Regulation of Other Endothelial Cell-Specific Promoters in Tumor Xenografts

As described herein, the Flt-1 promoter, but not the vWF or Tie-2 promoter, directs gene expression in the vascular beds of tumors. To identify other endothelial cell-specific transgenes that whose expression is induced during tumor angiogenesis, tumors (Lewis Lung Carcinoma and B16-F1 melanoma cells) are implanted into established lines of Hprt-targeted mice that carry endothelial cell-specific transgenes, including eNOS (−1600-+10) (endothelial neuronal nitric oxide (eNO) synthase), Egr-1 (−1200) (Early growth response factor-1), VCAM-1 (−1000) (vascular cell adhesion molecule-1), ICAM-1 (intercellular adhesion molecule 1) and Flk-1 (−1100). These lines have already been generated and are now backcrossed for over 7 generations to C57BL/6. Tumor whole mounts and cryosections are processed for LacZ staining. If one or more of these promoters is activated in tumor endothelium, transient transfections in dermal microvascular endothelial cells will be employed to determine whether the promoter is responsive to tumor cell-conditioned medium. If there is induction, the responsible cis-regulatory elements will be mapped in vitro and then tested in vivo as described above for the endocan promoter. Initially, the focus will be on those transcriptional control elements that are likely to be important for basal and/or inducible expression (i.e. Ets-binding sites). The information from these studies will provide a powerful foundation for the generation of non-naturally occurring tumor-inducible promoters.

### Mapping the Tumor Endothelial Cell-Specific Pathway that Regulates the Flt-1 Gene

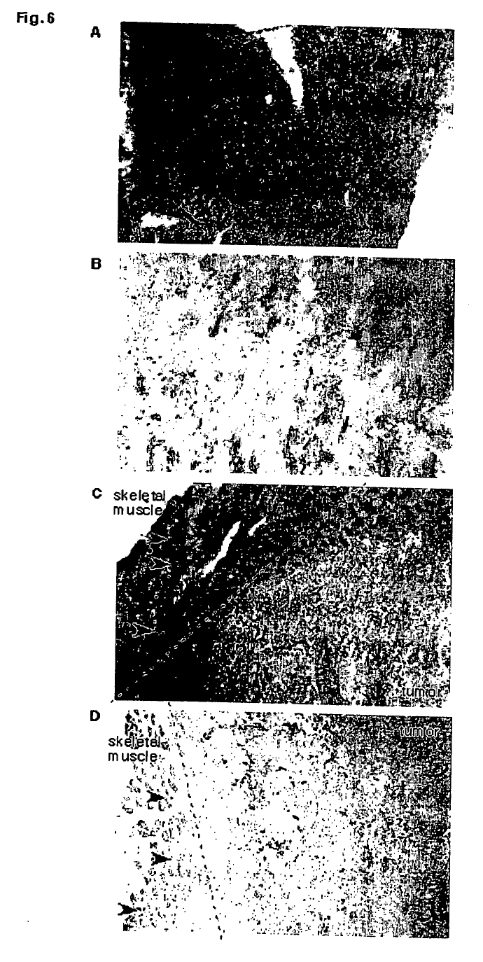

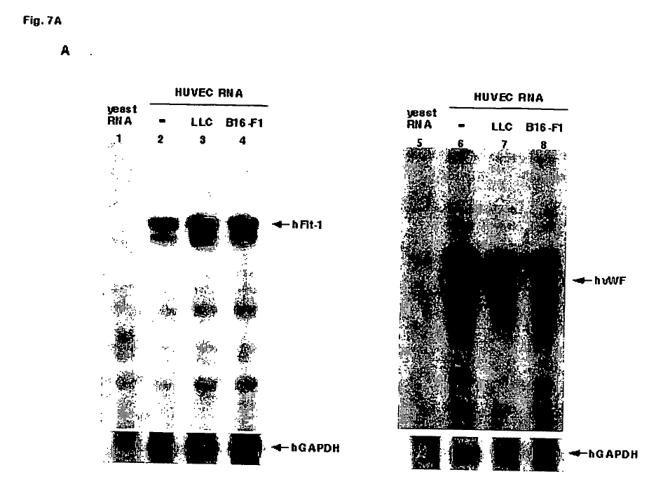

As detailed above, the Flt-1 promoter directs expression in the neovasculature of tumor xenografts. In contrast, the vWF or Tie-2 promoters, while active in other vascular beds, were not expressed in tumor endothelium. FIGS. 6A-6D show tumor sections from Flt-1 and vWF mice. The addition of tumor cell-conditioned medium or VEGF to HUVECs resulted in reduced cell surface expression of Flt-1, but increased Flt-1 mRNA (Wang et al., J. Biol. Chem. 275:15905-15911, 2000). The effect of the tumor cell-conditioned medium was abrogated by pre-incubation with anti-VEGF antibodies. Other studies have shown that VEGF induces Flt-1 expression in endothelial cells (Barleon et al., Cancer Res. 57:5421-5425, 1997). In addition, hypoxia has been shown to induce the expression of Flt-1 in cultured endothelial cells (Gerber et al., J. Biol. Chem. 273:13313-13316, 1998). As shown in FIGS. 7A and 7B, our studies (transient transfections and co-culture) indicate that one or more tumor cell-derived soluble factors mediated the upregulation of Flt-1 expression during tumor angiogenesis.

Neutralizing antibodies are employed to identify the tumor-derived factors. Flt-1 promoter reporter gene constructs are transfected into primary human endothelial cells and the cells are then incubated in the absence or presence of antibodies to VEGF, bFGF, Platelet-derived growth factor-BB (PDGF-BB) and epidermal growth factor (EGF). In parallel experiments, the intracellular signaling pathway is mapped with an array of chemical inhibitors and dominant negative adenoviruses that have been used extensively in other studies. Finally, and most importantly, 5′ and internal deletions and mutations are employed to dissect the Flt-1 promoter elements that are responsible for mediating the response to tumor cell conditioned medium. The immediate upstream promoter of the human Fit-1 gene contains five Ets-binding motifs, two GC boxes and a cAMP response element (CRE). Flt-1 expression was shown to be mediated by a cooperative interaction between the CRE at −83 and an Ets motif at −54, relative to the start site of transcription (Wakiya et al., J. Biol. Chem. 271:30823-30828, 1996). It is noteworthy that tumor endothelial cells have been shown to express increased levels of Ets-1 (Lelievre et al., Int. J. Biochem. Cell. Biol. 33:391-407, 2001; Valter et al., Cancer Res. 59:5608-5614, 1999). These observations suggest that the Ets motif at −54 may play a critical role in mediating expression of Flt-1 during tumor angiogenesis. The importance of the DNA element identified in our in vitro studies will be verified in vivo by targeting the mutant/deletant construct to the Hprt locus of mice.

### Novel Non-Naturally Occurring Promoters to Specifically Target Tumor Endothelium

As described above, the invention provides compositions and methods for identifying nucleic acid sequences that direct the expression of a heterologous nucleic acid sequence specifically to the endothelium of tumor blood vessels. To that end, a nucleic acid sequence that is required for the induction of gene expression in response to a tumor secreted factor is operably-linked to a minimal core promoter (the minimal core promoter may be 5′ or 3′ to the responsive element. The capacity of such a neoplasm endothelium-specific promoter to transduce neoplasm-derived signals is then tested in transient transfections assays, followed by Hprt-locus targeting in mice. The following cis-regulatory elements will be tested: 1) the neoplasm-derived signals responsive sites of the endocan gene as defined above; 2) the tumor-responsive elements of transgenes that direct expression during tumor angiogenesis, including Flt-1+/−eNOS, Egr-1, VCAM-1, ICAM-1 and Flk-1, as described above (these promoter elements are likely to consist of some combination of Ets, NF-κB, CRE, SP1, GATA and/or SRE motifs); and 3) neoplasm-derived signals responsive DNA elements that may be predicted to be induced in the environment of a tumor, including hypoxia response elements. As a minimal core promoter, a short 400-bp fragment of the Tie-2 gene (GenBank accession number U53603) is employed. When targeted to the Hprt locus of mice, this short fragment does not express in any blood vessel or tissue. When combined with a small intronic enhancer element from the first intron of the Tie-2 gene (GenBank accession number U85629), it expresses widely in the endothelium In other words, this minimal promoter is poised to express in an endothelial cell-specific manner when coupled to appropriate DNA sequence(s).

### Methods for Delivering Therapeutic Polypeptides to Neoplasm Endothelial Cells

The stable expression of a therapeutic polypeptide in a stably transformed endothelial cell is one therapeutic approach for ameliorating a neoplasm. Heterologous nucleic acid molecules encoding, for example, anti-angiogenic proteins can be delivered to a neoplasm endothelial cell. Expression of anti-angiogenic proteins in such neoplasm endothelial cells can inhibit the growth, proliferation, maturation, or formation of new blood vessels and thereby inhibit neoplasm growth. The nucleic acid molecules must be delivered to those cells in a form in which they can be taken up by the cells and so that sufficient levels of protein can be produced to decrease, for example, neoplasm blood cell growth.

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a full length gene (e.g., an anti-angiogenic polypeptide), or a portion thereof, can be cloned into a retroviral vector and expression can be driven from its endogenous promoter, from the retroviral long terminal repeat, or from a promoter specifically expressed in a target cell type of interest (e.g., a neoplasm endothelial cell). Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). Most preferably, a viral vector is used to administer the gene of interest to a neoplasm endothelial cell.

Non-viral approaches can also be employed for the introduction of therapeutic nucleic acids to a cell of a patient having a neoplasm. For example, a nucleic acid molecule can be introduced into a cell by administering the nucleic acid in the presence of lipofection (Felgner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of

Biological Chemistry 264:16985, 1989), or by micro-injection under surgical conditions (Wolff et al., Science 247:1465, 1990). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue.

cDNA expression for use in gene therapy methods can be directed from any suitable promoter (e.g., an endocan promoter, Flt-1 promoter, or other neoplasm endothelial promoter identified using the methods described herein), and regulated by any appropriate mammalian regulatory element. For example, if desired, an enhancers known to preferentially direct gene expression in a neoplasm endothelial cell, (e.g., the 300 base pair Tie-2 intronic enhancer element described herein) can be used to direct the expression of a nucleic acid. Such enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Another therapeutic approach included in the invention involves administration of a recombinant polypeptide, either directly to the site of a potential or actual neoplasm or systemically (for example, by any conventional recombinant protein administration technique). The dosage of the administered protein depends on a number of factors, including the size and health of the individual patient. For any particular subject, the specific dosage regimes should be adjusted over time according to the individual need and the professional judgement of the person administering or supervising the administration of the compositions. Generally, between 0.1 mg and 100 mg, is administered per day to an adult in any pharmaceutically acceptable formulation.

Inhibitory Nucleobase Oligomers

The present invention features the use of inhibitory nucleobase oligomers to downregulate the expression of endocan mRNA or proteins. Inhibitory nucleobase oligomers (e.g., double stranded RNA (dsRNA), short interfering RNA (siRNA), antisense RNA, short hairpin RNA (shRNA), and mimetics thereof) decrease the expression of target genes. Using the nucleic acid sequence of an endocan nucleic acid, inhibitory oligonucleotides (e.g., nucleic acids or nucleobase oligomers) that decrease endocan expression may be identified. Inhibitory oligonucleotides targeting endocan are useful for a variety of applications, including RNAi therapies.

RNA interference (RNAi) is a mechanism of post-transcriptional gene silencing (PTGS) in which double-stranded RNA (dsRNA) corresponding to a gene or mRNA of interest is introduced into an organism resulting in the degradation of the corresponding mRNA. In the RNAi reaction, both the sense and anti-sense strands of a dsRNA molecule are processed into small RNA fragments or segments ranging in length from 21 to 23 nucleotides (nt) and having 2-nucleotide 3′ tails. Alternatively, synthetic dsRNAs, which are 21 to 23 nt in length and have 2-nucleotide 3′ tails, can be synthesized, purified and used in the reaction. These 21 to 23 nt dsRNAs are known as “guide RNAs” or “short interfering RNAs” (siRNAs).

The siRNA duplexes then bind to a nuclease complex composed of proteins that target and destroy endogenous mRNAs having homology to the siRNA within the complex. Although the identity of the proteins within the complex remains unclear, the function of the complex is to target the homologous mRNA molecule through base pairing interactions between one of the siRNA strands and the endogenous mRNA. The mRNA is then cleaved approximately 12 nt from the 3′ terminus of the siRNA and degraded. In this manner, specific genes can be targeted, and the mRNAs transcribed from them degraded, thereby resulting in a loss of protein expression from the targeted gene.

The specific requirements and modifications of dsRNA are described in PCT application number WO01/75164 (incorporated herein by reference). While dsRNA molecules can vary in length, it is most preferable to use siRNA molecules which are 21- to 23-nucleotide dsRNAs with characteristic 2- to 3-nucleotide 3′ overhanging ends typically either (2′-deoxy)thymidine or uracil. The siRNAs typically comprise a 3′ hydroxyl group. Single stranded siRNA as well as blunt ended forms of dsRNA can also be used. In order to further enhance the stability of the RNA, the 3′ overhangs can be stabilized against degradation. In one such embodiment, the RNA is stabilized by including purine nucleotides, such as adenosine or guanosine. Alternatively, substitution of pyrimidine nucleotides by modified analogs, e.g., substitution of uridine 2-nucleotide overhangs by (2′-deoxy)thymide is tolerated and does not affect the efficiency of RNAi. The absence of a 2′ bydroxyl group significantly enhances the nuclease resistance of the overhang in tissue culture medium.

Alternatively siRNA can be prepared using any of the methods set forth in PCT number WO01/75164 (incorporated herein by reference) or using standard procedures for in vitro transcription of RNA and dsRNA annealing procedures as described by Elbashir et al. (Genes & Dev. 15:188-200, 2001). siRNAs are also obtained as described by Elbashir et al. By incubation of dsRNA that corresponds to a sequence of the target gene in a cell-free Drosophila lysate from syncytial blastoderm Drosophila embryos under conditions in which the dsRNA is processed to generate siRNAs of about 21 to about 23 nucleotides, which are then isolated using techniques known to those of skill in the art. For example, gel electrophoresis can be used to separate the 21-23 nt RNAs and the RNAs can then be eluted from the gel slices. In addition, chromatography (e.g. size exclusion chromatography), glycerol gradient centrifugation, and affinity purification with antibody can be used to isolate the 21 to 23 nt RNAs.

In the present invention, the dsRNA, or siRNA, is complementary to the mRNA sequence of an endocan mRNA and can reduce or inhibit expression of endocan. Preferably the decrease in endocan protein expression is at least 10% relative to cells treated with a control dsRNA or siRNA, more preferably 25%, and most preferably at least 50%. Methods for assaying levels of protein expression are also well known in the art and include western blotting, immunoprecipitation, and ELISA.

In the present invention, the nucleic acids used include any modification that enhances the stability or function of the nucleic acid in any way. Examples include modifications to the phosphate backbone, the internucleotide linkage, or to the sugar moiety.

siRNA

Methods for designing siRNAs are known to the skilled artisan. (See, for example, Dykxhoorn Nature Rev Mol Cell Biol 4:457-467, 2003; Paddison et al. Genes Dev. 16:948-958, 2002; Paddison et al., Proc Natl Acad Sci USA. 99:1443-1448, 2002; Sohail et al., Nucleic Acids Res. 31:e38, 2003; Yu et al., Proc Natl Acad Sci USA. 99:6047-6052, 2002.) While various parameters are used to identify promising RNAi targets, the most effective siRNA and shRNA candidate sequences are identified by empirical testing.

siRNA Delivery

For some applications, a plasmid is used to deliver an endocan inhibitory nucleobase oligomer, such as a double stranded RNA, siRNA, or shRNA, as a transcriptional product. In such embodiments, the plasmid is designed to include a coding sequence for each of the sense and antisense strands of an endocan RNAi construct. The coding sequences can be the same sequence, e.g., flanked by inverted promoters, or can be two separate sequences each under the transcriptional control of separate promoters. After the coding sequence is transcribed, the complementary endocan RNA transcripts base pair to form a double-stranded RNA. PCT application WO01/77350 describes an exemplary vector for bi-directional transcription of a transgene to yield both sense and antisense RNA transcripts of the same transgene in a eukaryotic cell.

Methods for the production and therapeutic administration of siRNAs for in vivo therapies are described in U.S. Patent Application Publications: 20030180756, 20030157030, and 20030170891. Methods describing the successful in vivo use of siRNA are described by Song et al. (Nature Medicine 9: 347-351, 2003.

Administration to cells of endocan inhibitory nucleic acids, or vectors encoding such nucleic acids, can be carried out by any standard method. For example, an endocan inhibitory nucleic acid or a vector encoding an endocan inhibitory nucleic acid can be introduced in vivo by lipofection. Liposomes for encapsulation and transfection of nucleic acids in vitro may be used. For some applications, synthetic cationic lipids can be used to prepare liposomes for in vivo transfection (Felgner et. al., Proc. Natl. Acad. Sci. USA 84:7413-7417, 1987; See also, Mackey, et al., Proc. Natl. Acad. Sci. USA 85:8027-8031, 1988; Ulmer et al., Science 259:1745-1748, 1993). The use of cationic lipids may promote encapsulation of negatively charged nucleic acids, and also promote fusion with negatively charged cell membranes (Felgner and Ringold, Science 337:387-388, 1989). Particularly useful lipid compounds and compositions for transfer of nucleic acids are described in WO95/18863, WO96/17823, and in U.S. Pat. No. 5,459,127. Other molecules are also useful for facilitating transfection of a nucleic acid in vivo, such as a cationic oligopeptide (e.g., WO95/21931), peptides derived from DNA binding proteins (e.g., WO96/25508), or a cationic polymer (e.g., WO95/21931).

Alternatively, the nucleic acid can be introduced into the cells by any means appropriate for the vector employed. Many such methods are well known in the art (Sambrook et al., supra, and Watson et al., Recombinant DNA, Chapter 12, 2d edition, Scientific American Books, 1992). Recombinant vectors can be transferred by methods such as calcium phosphate precipitation, electroporation, liposome-mediated transfection, gene gun, microinjection, viral capsid-mediated transfer, polybrene-mediated transfer, protoplast fusion, etc. For a review of the procedures for liposome preparation, targeting and delivery of contents, see Mannino et al. (Bio Techniques 6:682-690, 1988), Felgner et al. (Bethesda Res. Lab. Focus 11:21, 1989), and Maurer (Bethesda Res. Lab. Focus 11:25, 1989). A variety of methods are available for transfection, or introduction, of dsRNA or nucleic acids into mammalian cells. For example, there are several commercially available transfection reagents including but not limited to: TransIT-TKO3 (Mirus, Cat. # MIR 2150), Transmessenger3 (Qiagen, Cat. # 301525), and Oligofectamine3 (Invitrogen, Cat. # MIR 12252-011). Protocols for each transfection reagent are available from the manufacturer.

Transfer of the recombinant vector (either plasmid or viral vectors) can be accomplished in vivo by a variety of methods, e.g., by intravenous delivery. U.S. Pat. Nos. 5,830,879 and 6,258,787 describe additional techniques for delivery of nucleic acid to target cells or tissues. Gene delivery using adenoviral vectors or adeno-associated vectors (AAV) can also be used. Adenoviruses are present in a large number of animal species, are not very pathogenic, and can replicate equally well in dividing and quiescent cells. As a general rule, adenoviruses used for gene delivery are lacking one or more genes required for viral replication. Replication-defective recombinant adenoviral vectors used for the delivery of nucleic acids can be produced in accordance with art-known techniques (see Quantin et al., Proc. Natl. Acad. Sci. USA 89:2581-2584, 1992; Stratford-Perricadet et al., J. Clin. Invest. 90:626-630, 1992; and Rosenfeld et al., Cell 68:143-155, 1992).

Once transferred, the nucleic acid is expressed by the cells at the site of the neoplasm for a period of time sufficient to reduce levels of endocan. Because the vectors containing the nucleic acid are not normally incorporated into the genome of the cells, expression of the molecule of interest takes place for only a limited time. Typically, the molecule is expressed at therapeutic levels for about two days to several weeks, preferably for about one to two weeks. The inhibiting molecule (e.g., antisense nucleic acid, dsRNA, or siRNA specific to endocan mRNA) can be reapplied or readministered to provide additional periods of expression of the inhibiting molecule

It is also possible to introduce an endocan inhibitory nucleic acid or an expression vector encoding such a nucleic acid in vivo as a naked DNA. Methods for formulating and administering naked DNA to mammalian tissue are disclosed in U.S. Pat. Nos. 5,580,859 and 5,589,466.

The inhibitory nucleic acids and nucleobase oligomers of the invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The nucleobase oligomers of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The nucleobase oligomers of the invention encompass any pharmaceutically acceptable salts, esters, or salts of such esters, or any other compound that, upon administration to an animal, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention can be prepared as SATE [(S-acetyl-2-thioethyl) phosphate] derivatives according to the methods disclosed in PCT publication Nos. WO 93/24510 or WO 94/26764.

The term “pharmaceutically acceptable salts” refers to salts that retain the desired biological activity of the parent compound and do not impart undesired toxicological effects thereto. Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., J. Pharma Sci., 66:1-19, 1977). The base addition salts of acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides and other nucleobase oligomers, suitable pharmaceutically acceptable salts include (i) salts formed with cations such as sodium, potassium, ammonium, magnesium, calcium, polyamines such as spermine and spermidine, etc.; (ii) acid addition salts formed with inorganic acids, for example hydrochloric acid, hydrobromic acid, sulfuric acid, phosphoric acid, nitric acid and the like; (iii) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (iv) salts formed from elemental anions such as chlorine, bromine, and iodine.

The present invention also includes pharmaceutical compositions and formulations that include the nucleobase oligomers of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral, or parenteral. Parenteral administration includes intravenous, intraarterial, subcutaneous, intraperitoneal, or intramuscular injection or infusion; or intracranial, e.g., intrathecal or intraventricular, administration.

Because inhibitory nucleic acids may be substrates for nuclease degradation, modified or substituted inhibitory nucleic acids are often preferred because of properties such as, for example, enhanced cellular uptake and increased stability in the presence of nucleases.

Modified Nucleobase Oligomers

An endocan inhibitory nucleic acid or nucleobase oligomer may include modifications that increase nuclease resistance or that enhance the activity, cellular distribution or cellular uptake of the oligonucleotide. In various embodiments, an endocan oligomeric mimetic contains novel groups in place of the sugar, the backbone, or both. The base units are maintained to allow hybridization with an appropriate nucleic acid target compound.

Specific examples of some preferred endocan nucleic acids envisioned for this invention may contain phosphorothioates, phosphotriesters, methyl phosphonates, short chain alkyl or cycloalkyl intersugar linkages or short chain heteroatomic or heterocyclic intersugar linkages. Most preferred are those with CH₂—NH—O—CH₂, CH₂—N(CH₃)—O—CH₂, CH₂—O—N(CH₃)—CH₂, CH₂—N(CH₃)—N(CH₃)—CH₂ and O—N(CH₃)—CH₂—CH₂ backbones (where phosphodiester is O—P—O—CH₂). Also preferred are oligonucleotides having morpholino backbone structures (Summerton, J. E. and Weller, D. D., U.S. Pat. No. 5,034,506). In other preferred embodiments, such as the protein-nucleic acid (PNA) backbone, the phosphodiester backbone of the oligonucleotide may be replaced with a polyamide backbone, the bases being bound directly or indirectly to the aza nitrogen atoms of the polyamide backbone (P. E. Nielsen, M. Egholm, R. H. Berg, O. Buchardt, Science 199, 254, 1497). Other preferred endocan oligonucleotides may contain alkyl and halogen-substituted sugar moieties comprising one of the following at the 2′ position: OH, SH, SCH₃, F, OCN, O(CH₂)ₙNH₂ or O(CH₂)ₙCH₃, where n is from 1 to about 10; C₁ to C₁₀ lower alkyl, substituted lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a conjugate; a reporter group; an intercalator; a group for improving the pharmacokinetic properties of an oligonucleotide; or a group for improving the pharmacodynamic properties of an oligonucleotide and other substituents having similar properties. Endocan oligonucleotides may also have sugar mimetics such as cyclobutyls in place of the pentofuranosyl group.

In other preferred embodiments, an endocan oligomer may include at least one modified base form. Some specific examples of such modified bases include 2-(amino)adenine, 2-(methylamino)adenine, 2-(imidazolylalkyl)adenine, 2-(aminoalklyamino)adenine, or other heterosubstituted alkyladenines. In one embodiment, an endocan oligomer includes one or more G-clamp nucleotides. A G-clamp nucleotide is a modified cytosine analog having a modification that confers the ability to hydrogen bond both Watson-Crick and Hoogsteen faces of a complementary guanine within a duplex, see for example Lin and Matteucci, 1998, J. Am. Chem. Soc., 120, 8531-8532. A single G-clamp analog substitution within an oligomer can result in substantially enhanced helical thermal stability and mismatch discrimination when hybridized to complementary oligonucleotides. In another embodiment, endocan nucleic acid molecules of the invention include one or more LNA “locked nucleic acid” nucleotides such as a 2′,4′-C mythylene bicyclo nucleotide (see for example Wengel et al., International PCT Publication No. WO 00/66604 and WO 99/14226).

In other embodiments, an endocan oligomer contains one or more moieties or conjugates which enhance the activity, cellular distribution or cellular uptake of the oligonucleotide. The compounds of the invention can include conjugate groups covalently bound to functional groups such as primary or secondary hydroxyl groups. Conjugate groups include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the properties of an oligonucleotide include groups that improve oligomer uptake, enhance oligomer resistance to degradation, and/or strengthen sequence-specific hybridization with RNA, improve oligomer uptake, distribution, metabolism, or excretion. Conjugate moieties include, but are not limited to, lipid moieties such as a cholesterol moiety, cholic, a thioether, e.g., hexyl-S-tritylthiol, athiocholesterol, analiphatic chain, a phospholipid, e.g., di-hexadecyl-rac-glycerol or triethyl-ammonium 1,2-di-O-hexadecyl-rac-glyc-ero-3-H-phosphonate, a polyamine or a polyethylene glycol chain, or adamantane acetic acid, a palmityl moiety, or an octadecylamine orhexylamino-carbonyl-oxycholesterol moiety. Methods for the preparation of such oligonucleotide conjugates are standard in the art, and include, but are not limited to exonuclease resistant terminally substituted oligonucleotides, which are described in U.S. Pat. No. 5,245,022; oligonucleotide-enzyme conjugates, which are described in U.S. Pat. No. 5,254,469; boronated phosphoramidate conjugates, which are described in U.S. Pat. No. 5,272,250; detectably tagged oligomers, which are described in U.S. Pat. No. 5,317,098; oligomer protein conjugates, which are described in U.S. Pat. No. 5,391,723; and steroid modified oligomers, which are described in U.S. Pat. No. 5,416,203. Other oligonucleotide conjugates are described in, for example, in U.S. Pat. Nos. 5,258,506; 5,262,536; 5,292,873; 5,371,241, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

Endocan oligomers may also include nucleobase (often referred to in the art simply as “base”) modifications or substitutions. “Unmodified” or “natural” nucleotides include the purine bases adenine (A) and guanine (G), and the pyrimidine bases thymine (T), cytosine (C) and uracil (U). Modified nucleotides are known in the art, and are described in U.S. Pat. No. 3,687,808, The Concise Encyclopedia Of Polymer Science And Engineering, pages 858-859, Kroschwitz, J. I., ed. John Wiley & Sons, 1990, Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B. ed., CRC Press, 1993. Modified nucleobases are particularly useful for increasing the binding affinity of the oligomeric compounds of the invention. These modified nucleobases include, but are not limited to, 5-substituted pyrimidines, 6-azapyrimidines and N-2, N-6 and 0-6 substituted purines, including 2-aminopropyladenine, 5-propynyluracil and S-propynylcytosine. 5-methylcytosine substitutions.

Oligonucleotide Backbones

At least two types of oligonucleotides induce the cleavage of RNA by Rnase H: oligodeoxynucleotides with phosphodiester (PO) or phosphorothioate (PS) linkages. Although 2′-OMe-RNA sequences exhibit a high affinity for RNA targets, these sequences are not substrates for RNase H. A desirable oligonucleotide is one based on 2′-modified oligonucleotides containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅₀. This modification also increases the nuclease resistance of the modified oligonucleotide. Peptide Nucleic Acids (PNA) may also be employed.

Locked Nucleic Acids

Locked nucleic acids (LNA) are nucleotide analogs that can be employed in the present invention. LNA contain a 2′O, 4′-C methylene bridge that restrict the flexibility of the ribofuranose ring of the nucleotide analog and locks it into the rigid bicyclic N-type conformation. LNA show improved resistance to certain exo- and endonucleases and activate RNAse H, making them suitable for use in methods described herein. LNA can be incorporated into almost any oligonucleotide. Moreover, LNA-containing oligonucleotides can be prepared using standard phosphoramidite synthesis protocols. Additional details regarding LNA can be found in PCT publication WO99/14226, hereby incorporated by reference.

Arabinonucleic Acids

Arabinonucleic acids (ANA) can also be employed in the methods and reagents of the present invention. ANA are based on D-arabinose sugars instead of the natural D-2′-deoxyribose sugars. Underivatized ANA analogs have similar binding affinity for RNA as phosphorothioates. When the arabinose sugar is derivatized with fluorine (2′ F-ANA), an enhancement in binding affinity results, and selective hydrolysis of bound RNA occurs efficiently in the resulting ANA/RNA and F-ANA/RNA duplexes. These analogs can be made stable in cellular media by a derivatization at their termini with simple L sugars.

Screening Assays

As discussed above, endocan nucleic acids and polypeptides are expressed in neoplasm endothelium. Based on this discovery, screening assays to identify compounds that decrease the expression of an endocan polypeptide or nucleic acid sequence are useful for identifying therapeutic compounds for the treatment of a neoplasm. The method of screening may involve high-throughput techniques. In addition, these screening techniques may be carried out in cultured cells or in animals, such as mice.

Any number of methods are available for carrying out such screening assays. In one example, candidate compounds are added at varying concentrations to the culture medium of cultured cells expressing an endocan nucleic acid sequence. Endocan gene expression is then measured, for example, by standard Northern blot analysis (Ausubel et al., supra) or RT-PCR, using any appropriate fragment prepared from the nucleic acid molecule as a hybridization probe. The level of endocan gene expression in the presence of the candidate compound is compared to the level measured in a control culture medium lacking the candidate molecule. A compound that inhibits endocan expression is considered useful in the invention; such a molecule may be used, for example, as a therapeutic to treat a neoplasm.

In another example, the effect of candidate compounds is measured at the level of endocan polypeptide production using the same general approach and standard immunological techniques, such as Western blotting or immunoprecipitation with an antibody specific for an endocan polypeptide. For example, immunoassays may be used to detect or monitor the expression of at least one of the polypeptides of the invention in an organism. Polyclonal or monoclonal antibodies (produced as described above) that are capable of binding to such a polypeptide may be used in any standard immunoassay format (e.g., ELISA, Western blot, or RIA assay) to measure the level of the polypeptide. In another example, endocan polypeptide expression is detected by fusing the endocan polypeptide to a detectable reporter. A compound that reduces the expression of the polypeptide is considered particularly useful. Again, such a molecule may be used, for example, as a therapeutic to treat a neoplasm.

In yet another working example, candidate compounds are screened for those that specifically bind to and antagonize an endocan polypeptide. The efficacy of such a candidate compound is dependent upon its ability to interact with endocan or a functional equivalent thereof. Such an interaction can be readily assayed using any number of standard binding techniques and functional assays (e.g., those described in Ausubel et al., supra). For example, a candidate compound may be tested in vitro for interaction and binding with a polypeptide of the invention and its ability to treat a neoplasm may be assayed by any standard assay (e.g., those described herein).

In one particular working example, a candidate compound that binds to an endocan polypeptide may be identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for the endocan polypeptide is identified on the basis of its ability to bind to the endocan polypeptide and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography). In addition, these candidate compounds may be tested for their ability to treat a neoplasm (e.g., as described herein). Compounds isolated by this approach may also be used, for example, as therapeutics to delay or ameliorate human diseases associated with the expression or overexpression of a gene. Compounds that are identified as binding to an endocan polypeptide or an endocan active site with an affinity constant less than or equal to 10 mM are considered particularly useful in the invention.

Potential antagonists include organic molecules, peptides, peptide mimetics, polypeptides, nucleic acids, and antibodies that bind to an endocan nucleic acid sequence or polypeptide of the invention and thereby decrease its nuclease activity. Potential antagonists also include small molecules that bind to and occupy the active site of the polypeptide thereby preventing binding to cellular binding molecules, such that normal biological activity is prevented.

Each of the endocan DNA sequences provided herein may also be used in the discovery and development of compounds for the treatment of a neoplasm. The encoded endocan protein, upon expression, can be used as a target for the screening of drugs that inhibit endocan biological activity

Additionally, the DNA sequences encoding the amino terminal regions of the encoded protein or Shine-Delgarno or other translation facilitating sequences of the respective mRNA can be used to construct inhibitory nucleic acid sequences to control the expression of the coding sequence of interest. Such sequences may be isolated by standard techniques (Ausubel et al., supra).

The antagonists of the invention may be employed, for instance, to prevent, or treat a neoplasm.

Optionally, compounds identified in any of the above-described assays may be confirmed as useful in delaying or ameliorating human diseases associated in either standard tissue culture methods or animal models and, if successful, may be used as therapeutics for treating a neoplasm.

Small molecules of the invention preferably have a molecular weight below 2,000 daltons, more preferably between 300 and 1,000 daltons, and most preferably between 400 and 700 daltons. It is preferred that these small molecules are organic molecules.

Test Compounds and Extracts

In general, compounds capable of treating a neoplasm by inhibiting endocan expression or biological activity are identified from large libraries of both natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity in inhibiting nuclease activity should be employed whenever possible.

When a crude extract is found to inhibit endocan expression or biological activity, further fractionation of the positive lead extract is necessary to isolate chemical constituents responsible for the observed effect. Thus, the goal of the extraction, fractionation, and purification process is the careful characterization and identification of a chemical entity within the crude extract that inhibits endocan. Methods of fractionation and purification of such heterogenous extracts are known in the art. If desired, compounds shown to be useful agents for inhibiting endocan expression or biological activity RNAi are chemically modified according to methods known in the art.

Assays for Gene and Protein Expression

The following methods can be used to evaluate protein or gene expression and determine efficacy for any of the above-mentioned methods for decreasing endocan protein levels.

Blood, serum, or neoplasm tissue from the patient is measured for endocan level. Methods used to measure protein levels include ELISA, western blotting, or radioimmunoassays using specific antibodies.

There are several art-known methods to assay for gene expression. Some examples include the preparation of RNA from the blood, serum, or neoplasm samples of the patient and the use of the RNA for northern blotting, PCR based amplification, or RNAse protection assays.

Use of Antibodies for Therapeutic Treatment

The elevated levels of endocan found in neoplasm samples suggest a role for endocan in regulating cell proliferation. The use of compounds, such as antibodies, that bind to endocan will likely suppress neoplasm growth by depleting free endocan that is available to stimulate cell proliferation and neoplasm growth. Growth of the neoplasm is thus suppressed.

A cocktail of the monoclonal antibodies of the present invention can be used to suppress effectively neoplasm growth. The cocktail may include as few as 2, 3, or 4 different antibodies or as many as 6, 8, or even more than 10 different antibodies. In addition, the antibodies of the present invention can be combined with any other medication used to treat neoplasms or the symptoms associated with a neoplasm.

Methods for the preparation and use of antibodies for therapeutic purposes are described in several patents including U.S. Pat. Nos. 6,054,297; 5,821,337; 6,365,157; and 6,165,464 and are incorporated herein by reference. Antibodies can be polyclonal or monoclonal; monoclonal antibodies are preferred.

Monoclonal antibodies, particularly those derived from rodents including mice, have been used for the treatment of various diseases; however, there are limitations to their use including the induction of a human anti-mouse immunoglobulin response that causes rapid clearance and a reduction in the efficacy of the treatment. For example, a major limitation in the clinical use of rodent monoclonal antibodies is an anti-globulin response during therapy (Miller et al., Blood 62:988-995 1983; Schroff et al., Cancer Res. 45:879-885, 1985).

The art has attempted to overcome this problem by constructing “chimeric” antibodies in which an animal antigen-binding variable domain is coupled to a human constant domain (U.S. Pat. No. 4,816,567; Morrison et al., Proc. Natl. Acad. Sci. USA 81:6851-6855, 1984; Boulianne et al., Nature 312:643-646, 1984; Neuberger et al., Nature 314:268-270, 1985). The production and use of such chimeric antibodies are described below.

Preparation of Antibodies

Monoclonal antibodies that specifically bind to endocan may be produced by methods known in the art. These methods include the immunological method described by Kohler et al. (Nature 256: 495-497, 1975), and Campbell (“Monoclonal Antibody Technology, The Production and Characterization of Rodent and Human Hybridomas” in Burdon et al., Eds., Laboratory Techniques in Biochemistry and Molecular Biology, Volume 13, Elsevier Science Publishers, Amsterdam, 1985), as well as the recombinant DNA method described by Huse et al. (Science 246:1275-1281, 1989).

Monoclonal antibodies may be prepared from supernatants of cultured hybridoma cells or from ascites induced by intra-peritoneal inoculation of hybridoma cells into mice. The hybridoma technique described originally by Kohler et al. (Eur. J. Immunol. 6:511-519, 1976) has been widely applied to produce hybrid cell lines that secrete high levels of monoclonal antibodies against many specific antigens.

The route and schedule of immunization of the host animal or cultured antibody-producing cells therefrom are generally in keeping with established and conventional techniques for antibody stimulation and production. Typically, mice are used as the test model; however, any mammalian subject including human subjects or antibody producing cells therefrom can be manipulated according to the processes of this invention to serve as the basis for production of mammalian, including human, hybrid cell lines.

After immunization, immune lymphoid cells are fused with myeloma cells to generate a hybrid cell line which can be cultivated and subcultivated indefinitely, to produce large quantities of monoclonal antibodies. For purposes of this invention, the immune lymphoid cells selected for fusion are lymphocytes and their normal differentiated progeny, taken either from lymph node tissue or spleen tissue from immunized animals. The use of spleen cells is preferred, since they offer a more concentrated and convenient source of antibody producing cells with respect to the mouse system. The myeloma cells provide the basis for continuous propagation of the fused hybrid. Myeloma cells are neoplasm cells derived from plasma cells. Murine myeloma cell lines can be obtained, for example, from the American Type Culture Collection (ATCC; Manassas, Va.). Human myeloma and mouse-human heteromyeloma cell lines have also been described (Kozbor et al., J. Immunol. 133:3001-3005, 1984; Brodeur et al., Monoclonal Antibody Production Techniques and Applications, Marcel Dekker, Inc., New York, pp. 51-63, 1987).

The hybrid cell lines can be maintained in vitro in cell culture media. Once the hybridoma cell line is established, it can be maintained on a variety of nutritionally adequate media such as hypoxanthine-aminopterin-thymidine (HAT) medium. Moreover, the hybrid cell lines can be stored and preserved in any number of conventional ways, including freezing and storage under liquid nitrogen. Frozen cell lines can be revived and cultured indefinitely with resumed synthesis and secretion of monoclonal antibody. The secreted antibody is recovered from tissue culture supernatant by conventional methods such as precipitation, ion exchange chromotography, affinity chromatography, or the like.

The antibody may be prepared in any mammal, including mice, rats, rabbits, goats, and humans. The antibody may be a member of one of the following immunoglobulin classes: IgG, IgM, IgA, IgD, or IgE, and the subclasses thereof, and preferably is an IgG antibody.

While the preferred animal for producing monoclonal antibodies is mouse, the invention is not so limited; in fact, human antibodies may be used and may prove to be preferable. Such antibodies can be obtained by using human hybridomas (Cole et al., Monoclonal Antibodies and Cancer Therapy, Alan R. Liss Inc., p. 77-96, 1985). In the present invention, techniques developed for the production of chimeric antibodies by splicing the genes encoding a mouse antibody molecule of appropriate antigen specificity together with genes encoding a human antibody molecule can be used (Morrison et al., Proc. Natl. Acad. Sci. 81:6851-6855, 1984; Neuberger et al., Nature 312:604-608, 1984; Takeda et al., Nature 314:452-454, 1985); such antibodies are within the scope of this invention and are described below.

As another alternative to the cell fusion technique, Epstein-Barr virus (EBV) immortalized B cells are used to produce the monoclonal antibodies of the present invention (Crawford et al., J. of Gen. Virol. 64:697-700, 1983; Kozbor et al., J. Immunol. 4:1275-1280, 1981; Kozbor et al., Methods in Enzymology 121:120-140, 1986). In general, the procedure consists of isolating Epstein-Barr virus from a suitable source, generally an infected cell line, and exposing the target antibody secreting cells to supernatants containing the virus. The cells are washed, and cultured in an appropriate cell culture medium. Subsequently, virally transformed cells present in the cell culture can be identified by the presence of the Epstein-Barr viral nuclear antigen, and transformed antibody secreting cells can be identified using standard methods known in the art. Other methods for producing monoclonal antibodies, such as recombinant DNA, are also included within the scope of the invention.

Preparation of Endocan Immunogens

Endocan may be used by itself as an immunogen, or may be attached to a carrier protein or to other objects, such as sepharose beads. Endocan may be purified from cells known to express it such as neoplasm endothelial cells. Additionally, nucleic acid molecules that encode endocan, or portions thereof, can be inserted into known vectors for expression in host cells using standard recombinant DNA techniques. Suitable host cells for endocan expression include baculovirus cells (e.g., Sf9 cells), bacterial cells (e.g., E. Coli), and mammalian cells (e.g., NIH3T3 cells).

In addition, peptides can be synthesized and used as immunogens. The methods for making antibody to peptides are well known in the art and generally require coupling the peptide to a suitable carrier molecule, such as serum albumin. Peptides include any amino acid sequence that is substantially identical to the endocan amino acid sequence corresponding to GenBank accession number X89426 or AJ249354. Preferably, the amino acid sequences are at least 60%, more preferably at least 85%, and most preferably at least 95% identical to the sequence of X89426 or AJ249354. The peptides can be commercially obtained or made using techniques well known in the art, such as, for example, the Merrifield solid-phase method (Science 232:341-347, 1985). The procedure may use commercially available synthesizers such as a Biosearth 9500 automated peptide machine, with cleavage of the blocked amino acids being achieved with hydrogen fluoride, and the peptides purified by preparative HPLC using a Waters Delta Prep 3000 instrument, on a 15-20 μm Vydac C4 PrepPAK column.

Functional Equivalents of Antibodies

The invention also includes functional equivalents of the antibodies described in this specification. Functional equivalents include polypeptides with amino acid sequences substantially identical to the amino acid sequence of the variable or hypervariable regions of the antibodies of the invention. Functional equivalents have binding characteristics comparable to those of the antibodies, and include, for example, chimerized, humanized and single chain antibodies as well as fragments thereof. Methods of producing such functional equivalents are disclosed in PCT Application WO 93/21319; European Patent Application No. 239,400; PCT Application WO 89/09622; European Patent Application No. 338,745; European Patent Application No. 332,424; and U.S. Pat. No. 4,816,567; each of which is incorporated herein by reference.

Chimerized antibodies preferably have constant regions derived substantially or exclusively from human antibody constant regions and variable regions derived substantially or exclusively from the sequence of the variable region from a mammal other than a human. Such humanized antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Methods for humanizing non-human antibodies are well known in the art (for reviews see Vaswani et al., Ann Allergy Asthma Immunol. 81:105-119, 1998 and Carter, Nature Reviews Cancer 1:118-129, 2001). Generally, a humanized antibody has one or more amino acid residues introduced into it from a source that is non-human. These non-human amino acid residues are often referred to as import residues, which are typically taken from an import variable domain. Humanization can be essentially performed following the methods known in the art (Jones et al., Nature 321:522-525, 1986; Riechmann et al., Nature 332:323-329, 1988; and Verhoeyen et al., Science 239:1534-1536, 1988), by substituting rodent CDRs or other CDR sequences for the corresponding sequences of a human antibody. Accordingly, such humanized antibodies are chimeric antibodies wherein substantially less than an intact human variable domain has been substituted by the corresponding sequence from a non-human species (see for example, U.S. Pat. No. 4,816,567). In practice, humanized antibodies are typically human antibodies in which some CDR residues and possibly some FR residues are substituted by residues from analogous sites in rodent antibodies (Presta, Curr. Op. Struct. Biol. 2:593-596, 1992).

Additional methods for the preparation of humanized antibodies can be found in U.S. Pat. Nos. 5,821,337, and 6,054,297, and Carter (supra), which are all incorporated herein by reference. The humanized antibody is selected from any class of immunoglobulins, including IgM, IgG, IgD, IgA and IgE, and any isotype, including IgG₁, IgG₂, IgG₃, and IgG₄. The humanized antibody may comprise sequences from more than one class or isotype, and selecting particular constant domains to optimize desired effector functions is within the ordinary skill in the art.

Human antibodies can also be produced using various techniques known in the art, including phage display libraries (Marks et al., J. Mol. Biol. 222:581-597, 1991 and Winter et al., Annu. Rev. Immunol. 12:433-455, 1994). The techniques of Cole et al. And Boerner et al. Are also useful for the preparation of human monoclonal antibodies (Cole et al., supra; Boerner et al., J. Immunol. 147: 86-95, 1991).

Suitable mammals other than a human include any mammal from which monoclonal antibodies may be made. Examples of mammals other than a human include, for example a rabbit, rat, mouse, horse, goat, or primate; a mouse is preferred.

Functional equivalents of antibodies also include single-chain antibody fragments, also known as single-chain antibodies (scFvs). Single-chain antibody fragments are recombinant polypeptides which typically bind antigens or receptors; these fragments contain at least one fragment of an antibody variable heavy-chain amino acid sequence (V_{H}) tethered to at least one fragment of an antibody variable light-chain sequence (V_{L}) with or without one or more interconnecting linkers. Such a linker may be a short, flexible peptide selected to assure that the proper three-dimensional folding of the V_{L} and V_{H} domains occurs once they are linked so as to maintain the target molecule binding-specificity of the whole antibody from which the single-chain antibody fragment is derived. Generally, the carboxyl terminus of the V_{L} or V_{H} sequence is covalently linked by such a peptide linker to the amino acid terminus of a complementary V_{L} and V_{H} sequence. Single-chain antibody fragments can be generated by molecular cloning, antibody phage display library or similar techniques. These proteins can be produced either in eukaryotic cells or prokaryotic cells, including bacteria.

Single-chain antibody fragments contain amino acid sequences having at least one of the variable regions or CDRs of the whole antibodies described in this specification, but are lacking some or all of the constant domains of those antibodies. These constant domains are not necessary for antigen binding, but constitute a major portion of the structure of whole antibodies. Single-chain antibody fragments may therefore overcome some of the problems associated with the use of antibodies containing part or all of a constant domain. For example, single-chain antibody fragments tend to be free of undesired interactions between biological molecules and the heavy-chain constant region, or other unwanted biological activity. Additionally, single-chain antibody fragments are considerably smaller than whole antibodies and may therefore have greater capillary permeability than whole antibodies, allowing single-chain antibody fragments to localize and bind to target antigen-binding sites more efficiently. Also, antibody fragments can be produced on a relatively large scale in prokaryotic cells, thus facilitating their production. Furthermore, the relatively small size of single-chain antibody fragments makes them less likely than whole antibodies to provoke an immune response in a recipient.

Functional equivalents further include fragments of antibodies that have the same or comparable binding characteristics to those of the whole antibody. Such fragments may contain one or both Fab fragments or the F(ab′)₂ fragment. Preferably the antibody fragments contain all six CDRs of the whole antibody, although fragments containing fewer than all of such regions, such as three, four or five CDRs, are also functional.

Further, the functional equivalents may be or may combine members of any one of the following immunoglobulin classes: IgG, IgM, IgA, IgD, or IgE, and the subclasses thereof.

Preparation of Functional Equivalents of Antibodies

Equivalents of antibodies are prepared by methods known in the art. For example, fragments of antibodies may be prepared enzymatically from whole antibodies. Preferably, equivalents of antibodies are prepared from DNA encoding such equivalents. DNA encoding fragments of antibodies may be prepared by deleting all but the desired portion of the DNA that encodes the full-length antibody.

DNA encoding chimerized antibodies may be prepared by recombining DNA substantially or exclusively encoding human constant regions and DNA encoding variable regions derived substantially or exclusively from the sequence of the variable region of a mammal other than a human. DNA encoding humanized antibodies may be prepared by recombining DNA encoding constant regions and variable regions other than the CDRs derived substantially or exclusively from the corresponding human antibody regions and DNA encoding CDRs derived substantially or exclusively from a mammal other than a human.

Suitable sources of DNA molecules that encode fragments of antibodies include cells, such as hybridomas, that express the full-length antibody. The fragments may be used by themselves as antibody equivalents, or may be recombined into equivalents, as described above.

The DNA deletions and recombinations described in this section may be carried out by known methods, such as those described in the published patent applications listed above.

Antibody Screening and Selection

Monoclonal antibodies are isolated and purified using standard art-known methods. For example, antibodies can be screened using standard art-known methods such as ELISA against the endocan antigen or western blot analysis. Examples of such techniques are described in Examples II and III of U.S. Pat. No. 6,365,157, herein incorporated by reference.

Therapeutic Uses of Antibodies

When used in vivo for the treatment of a neoplasm, the antibodies of the subject invention are administered to the patient in therapeutically effective amounts. Preferably, the antibodies are administered parenterally or intravenously by continuous infusion. The dose and dosage regimen depends upon the severity of the neoplasm, and the overall health of the patient. The amount of antibody administered is typically in the range of about 0.01 to about 10 mg/kg of patient weight.

For parenteral administration, the antibodies are formulated in a unit dosage injectable form (solution, suspension, emulsion) in association with a pharmaceutically acceptable parenteral vehicle. Such vehicles are inherently nontoxic, and non-therapeutic. Examples of such vehicles are water, saline, Ringer's solution, dextrose solution, and 5% human serum albumin. Nonaqueous vehicles such as fixed oils and ethyl oleate may also be used. Liposomes may be used as carriers. The vehicle may contain minor amounts of additives such as substances that enhance isotonicity and chemical stability, e.g., buffers and preservatives. The antibodies typically are formulated in such vehicles at concentrations of about 1 mg/ml to 10 mg/ml.

Neoplasm Therapy

Therapy for the treatment of a neoplasm may be provided wherever cancer therapy is performed: at home, the doctor's office, a clinic, a hospital's outpatient department, or a hospital. Treatment generally begins at a hospital so that the doctor can observe the therapy's effects closely and make any adjustments that are needed. The duration of the therapy depends on the kind of neoplasm being treated, the age and condition of the patient, the stage and type of the patient's disease, and how the patient's body responds to the treatment. Drug administration may be performed at different intervals (e.g., daily, weekly, or monthly). Therapy may be given in on-and-off cycles that include rest periods so that the patient's body has a chance to build healthy new cells and regain its strength.

Depending on the type of cancer and its stage of development, the therapy can be used to slow the spreading of the cancer, to slow the cancer's growth, to kill or arrest cancer cells that may have spread to other parts of the body from the original neoplasm, to relieve symptoms caused by the cancer, or to prevent cancer in the first place.

As used herein, the terms “cancer” or “neoplasm” or “neoplastic cells” mean a collection of cells multiplying in an abnormal manner. Cancer growth is uncontrolled and progressive, and occurs under conditions that would not elicit, or would cause cessation of, the multiplication of normal cells.

A nucleobase oligomer of the invention, anti-endocan antibody, or other negative regulator of endocan expression or biological activity, is administered within a pharmaceutically-acceptable diluent, carrier, or excipient, in unit dosage form. Conventional pharmaceutical practice may be employed to provide suitable formulations or compositions to administer the compounds to patients suffering from a disease that is caused by excessive cell proliferation. Administration may begin before the patient is symptomatic. Any appropriate route of administration may be employed, for example, administration may be parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for endocan modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for a disease or condition. The preferred dosage of a nucleobase oligomer of the invention is likely to depend on such variables as the type and extent of the disorder, the overall health status of the particular patient, the formulation of the compound excipients, and its route of administration.

As described above, if desired, treatment with a nucleobase oligomer of the invention may be combined with therapies for the treatment of proliferative disease, such as radiotherapy, surgery, or chemotherapy.

Diagnostics

The present invention also features diagnostic assays for diagnosing a neoplasm in a patient, monitoring the responsiveness of a neoplasm to therapy, and predicting patient prognosis. For such diagnostic methods, the level of endocan in a patient sample (e.g., blood or neoplasm sample) is measured and used as an indicator of the presence of a neoplasm, of a neoplasm's responsiveness to therapy, or of patient prognosis.

For the diagnosis of a neoplasm, endocan levels present in a patient sample (e.g., a blood sample) are compared to endocan levels present in a corresponding control sample (e.g., a blood sample from a healthy individual). The presence of increased endocan levels in the patient sample relative to the control indicates the presence of a neoplasm.

To monitor the response of a neoplasm to therapy, endocan levels are measured in a patient sample obtained from a patient undergoing treatment for a neoplasm. These levels are compared to endocan levels present in a reference sample obtained from the same patient prior to or at a defined time following the commencement of therapy. A decrease in endocan levels in the patient sample relative to the reference sample indicates that the patient's neoplasm is responding to treatment. No change in endocan levels or an increase in endocan levels in the patient sample relative to the reference sample indicates that the neoplasm has decreased responsiveness to therapy.

Patients whose endocan levels increase or fail to change in response to therapy have a poorer prognosis than patients having responsive neoplasms. While methods of neoplasia treatment vary depending on the type of neoplasia, the stage of neoplasia, and the patient's age, health, and physical condition, more aggressive treatment regimens will be used in patients having a poor prognosis. These include therapies having increased toxicity and those having an increased risk of adverse side-effects. Aggressive therapies are employed earlier and at higher doses in patients having a poor prognosis.

Endocan expression is measured by measuring gene or protein expression. Standard methods are used to measure endocan protein levels in blood serum or neoplasm samples while gene expression is measured in neoplasm samples. For endocan RNA measurements, such methods include PCR, RNA gel blot analysis using fluorescent- or radiolabeled probes, and microarray technology. PCR assays are the preferred method for measuring blood serum and neoplasm levels of endocan RNA. For endocan polypeptide measurements, such methods include radioimmunoassay, ELISA, western blotting using antibodies directed to endocan, and quantitative enzyme immunoassay techniques. ELISA assays are the preferred method for measuring blood serum and neoplasm levels of endocan polypeptide. An increase in blood serum or neoplasm level of endocan over time would indicate neoplasm progression, whereas a decrease in blood serum or neoplasm level of endocan over time would indicate neoplasm regression.

The invention also provides for a diagnostic test kit. For example, a diagnostic test kit may include antibodies to endocan, and means for detecting, and more preferably evaluating, binding between the antibodies and endocan. For detection, either the antibody or the endocan is labeled, and either the antibody or endocan is substrate-bound, such that the endocan-antibody interaction can be established by determining the amount of label attached to the substrate following binding between the antibody and endocan. A conventional ELISA is a common, art-known method for detecting antibody-substrate interaction and can be provided with the kit of the invention. Alternatively, a diagnostic test kit comprises endocan nucleic acid probes that hybridize to an endocan nucleic acid present in a patient sample. Such kits typically quantitate endocan RNA levels present in a sample. In one embodiment, the kit measures endocan RNA using PCR. Such a test kit will include endocan-specific probes. Optionally, the kit includes PCR reagents and materials for analyzing PCR products.

### OTHER EMBODIMENTS

From the foregoing description, it is apparent that variations and modifications may be made to the invention described herein to adopt it to various usages and conditions. Such embodiments are also within the scope of the following claims.

All publications mentioned in this specification are herein incorporated by reference to the same extent as if each independent publication or patent application was specifically and individually indicated to be incorporated by reference.

What is claimed is:

1. A method of diagnosing a patient as having a neoplasia, said method comprising detecting an endocan nucleic acid molecule or polypeptide in a patient sample, wherein detection of an endocan nucleic acid or polypeptide indicates that said patient has a neoplasia.

2. The method of claim 1, wherein said patient sample is a blood sample.

3. The method of claim 1, wherein said patient sample is a tissue sample.

4. The method of claim 1, wherein said method comprises detecting an increase in the level of expression of an endocan polypeptide in a patient sample relative to the level of endocan polypeptide present in a corresponding control sample from a normal individual.

5. The method of claim 4, wherein said level of expression is determined in an immunological assay.

6. The method of claim 5, wherein said level of expression is detected by ELISA.

7. The method of claim 1, wherein said patient is asymptomatic.

8. A method of assessing the responsiveness of a neoplasm to a treatment regimen, said method comprising determining the level of an endocan nucleic acid or polypeptide in a patient sample relative to the level in a reference sample, wherein an alteration in said nucleic acid or polypeptide level in said patient sample indicates the responsiveness of said neoplasm to a treatment regimen.

9. The method of claim 8, wherein said reference sample is derived from a healthy individual.

10. The method of claim 8, wherein said patient is being treated for a neoplasm.

11. The method of claim 8, wherein said reference sample is obtained from said patient prior to or during the course of said treatment regimen.

12. The method of claim 8, wherein said patient sample is a blood sample.

13. The method of claim 8, wherein said patient sample is a tissue sample.

14. The method of claim 8, wherein said alteration is an increase, and said increase indicates a decreased responsiveness of said neoplasm to a treatment regimen.

15. The method of claim 8, wherein said alteration is a decrease, and said decrease indicates an increased responsiveness of said neoplasm to a treatment regimen.

16. The method of claim 8, wherein said neoplasm is a renal cell carcinoma, a lung cancer, a glioma, or a breast carcinoma.

17. A method of determining the prognosis of a patient having a neoplasm, said method comprising detecting an alteration in the level of an endocan nucleic acid molecule or polypeptide in a patient sample relative to the level in a reference sample, wherein an alteration indicates the prognosis of said patient.

18. The method of claim 17, wherein said reference sample is obtained from said patient prior to or during the course of said treatment regimen.

19. The method of claim 17, wherein said alteration is an increase, and said increase indicates a poor prognosis.

20. The method of claim 17, wherein said alteration is a decrease, and said decrease indicates a good prognosis.

21. The method of claim 17, wherein said patient sample is a tissue sample or a blood sample.

22. The method of claim 17, wherein said level of expression is determined in an immunological or enzymatic assay.

23. The method of claim 17, wherein said neoplasm is a renal cell carcinoma, a lung cancer, a glioma, or a breast carcinoma.

24. A diagnostic kit for the detection of a neoplasm in a patient comprising an endocan nucleic acid or amino acid sequence, or a fragment thereof.

25. A diagnostic kit for the detection of a neoplasm in a patient comprising an anti-endocan antibody.

26-75. (canceled)
